(19) United States
(12) Patent Application Publication
(10) Pub. No.: US 2024/0212796 A1
(43) Pub. Date: Jun. 27, 2024

(54) REACTANT MOLECULE PREDICTION USING MOLECULE COMPLETION MODEL

(71) Applicant: Tencent Technology (Shenzhen) Company Limited, Shenzhen (CN)

(72) Inventors: Ziqiao MENG, Shenzhen (CN); Peilin ZHAO, Shenzhen (CN)

(73) Assignee: Tencent Technology (Shenzhen) Company Limited, Shenzhen (CN)

(21) Appl. No.: 18/597,636

(22) Filed: Mar. 6, 2024

Related U.S. Application Data
(63) Continuation of application No. PCT/CN2023/092036, filed on May 4, 2023.

(30) Foreign Application Priority Data
Jul. 14, 2022 (CN) 202210830979.X

(51) Int. Cl. G16C 20/20; G16C 20/50; G16C 20/70

(52) U.S. Cl. CPC G16C 20/20; G16C 20/50; G16C 20/70

(57) ABSTRACT

A method for predicting reactant molecules includes obtaining a product molecule, and selecting bonds of the product molecule to be broken to obtain a molecule to be completed, the product molecule defining a compound molecule of a reactant molecule to be predicted. The method further includes applying a molecule completion model to complete the molecule to be completed to obtain a completion result indicating a reactant molecule of the product molecule based on the molecule to be completed. The molecule completion model is obtained by training based on sample compound molecules and sample molecules to be completed obtained by masking sub-structures in the sample compound molecules.

### RELATED APPLICATIONS

This application is a continuation of International Application No. PCT/CN2023/092036, filed on May 4, 2023, which claims priority to Chinese Patent Application No. 202210830979.X, entitled “METHOD AND APPARATUS FOR PREDICTING REACTANT MOLECULES, METHOD AND APPARATUS FOR TRAINING MODELS, DEVICE, AND MEDIUM” filed on Jul. 14, 2022. The entire disclosures of the prior applications are hereby incorporated by reference in their entirety.

### FIELD OF THE TECHNOLOGY

Aspects of this disclosure relate to the technical field of artificial intelligence (AI), including a method and apparatus for predicting reactant molecules, a method and apparatus for training models, a device, and a medium.

### BACKGROUND OF THE DISCLOSURE

With the rise and rapid development of the AI technology, application scenarios in which a reactant molecule of a given product molecule is predicted are becoming increasingly widespread, such as chemical synthesis scenarios and drug preparation scenarios.

### SUMMARY

Aspects of this disclosure provide a method and apparatus for predicting reactant molecules, a method and apparatus for training models, a device, and a medium, which can be configured to improve the prediction reliability and accuracy of reactant molecules.

In an aspect, a method for predicting reactant molecules includes obtaining a product molecule, and selecting bonds of the product molecule to be broken to obtain a molecule to be completed, the product molecule defining a compound molecule of a reactant molecule to be predicted. The method further includes applying a molecule completion model to complete the molecule to be completed to obtain a completion result indicating a reactant molecule of the product molecule based on the molecule to be completed. The molecule completion model is obtained by training based on sample compound molecules and sample molecules to be completed obtained by masking sub-structures in the sample compound molecules.

In an aspect, a method for training molecule completion models includes obtaining a sample compound molecule and a sample molecule to be completed, the sample molecule to be completed being obtained by masking a sub-structure in the sample compound molecule. The method further includes determining a training loss based on the sample compound molecule, the sample molecule to be completed, and a molecule completion model, and updating model parameters of the molecule completion model based on the training loss to obtain a trained molecule completion model.

In an aspect, an apparatus for predicting reactant molecules includes processing circuitry configured to obtain a product molecule, and select bonds of the product molecule to be broken to obtain a molecule to be completed, the product molecule defining a compound molecule of a reactant molecule to be predicted. The processing circuitry is further configured to apply a molecule completion model to complete the molecule to be completed to obtain a completion result indicating a reactant molecule of the product molecule based on the molecule to be completed. The molecule completion model is obtained by training based on sample compound molecules and sample molecules to be completed obtained by masking sub-structures in the sample compound molecules.

According to the technical solutions provided in the aspects of this disclosure, the prediction process of the reactant molecule is implemented by the molecule completion model, and the molecule completion model is obtained by training based on the sample compound molecule and the sample molecule to be completed. The sample molecule to be completed is obtained by masking the sub-structure in the sample compound molecule, that is, the data for the training process of the molecule completion model is the data obtained on the basis of the sample compound molecule, this training process is a self-supervised training process based on the sample compound molecule, and this self-supervised training process does not require attention to whether the sample compound molecule is a compound in a known synthesis reaction, so that this self-supervised training process is not limited by the known synthesis reaction, and the molecule completion model trained by the training process has a stronger generalization ability, which is beneficial for expanding adaptation scenarios so as to improve the prediction reliability and prediction accuracy of the reactant molecule.

### BRIEF DESCRIPTION OF THE DRAWINGS

FIG. 1 is a schematic diagram of an implementation environment provided in an aspect of this disclosure.

FIG. 2 is a flowchart of a method for predicting reactant molecules provided in an aspect of this disclosure.

FIG. 3 is a schematic diagram of a representation form of a product molecule provided in an aspect of this disclosure.

FIG. 4 is a schematic diagram of two stages of a reactant molecule prediction process provided in an aspect of this disclosure.

FIG. 5 is a flowchart of a method for training molecule completion models provided in an aspect of this disclosure.

FIG. 6 is a schematic diagram of three cases of a masked sub-structure in a sample compound molecule provided in an aspect of this disclosure.

FIG. 7 is a schematic diagram of cutting different chemical bonds in a sample compound molecule provided in an aspect of this disclosure.

FIG. 8 is a schematic structural diagram of an initial chemical bond completion model provided in an aspect of this disclosure.

FIG. 9 is a schematic structural diagram of an initial atomic completion model provided in an aspect of this disclosure.

FIG. 10 is a schematic diagram of an apparatus for predicting reactant molecules provided in an aspect of this disclosure.

FIG. 11 is a schematic diagram of an apparatus for training molecule completion models provided in an aspect of this disclosure.

FIG. 12 is a schematic structural diagram of a server provided in an aspect of this disclosure.

FIG. 13 is a schematic structural diagram of a terminal provided in an aspect of this disclosure.

### DETAILED DESCRIPTION

In the related art, during prediction of a reactant molecule, first, a molecule to be completed is obtained according to a product molecule, then, a structure matched with the molecule to be completed is determined from a plurality of candidate structures, the matched structure is linked with the molecule to be completed, and the molecule obtained by linking is used as a predicted reactant molecule. The plurality of candidate structures are obtained by comparing difference structures between product molecules and reactant molecules in known synthesis reactions.

The foregoing method for predicting reactant molecules relies on a plurality of candidate structures extracted from known synthesis reactions, the generalization ability of the plurality of candidate structures is limited by the known synthesis reactions, the generalization ability is poor, and adaptation scenarios are relatively limited, so that the prediction reliability and prediction accuracy of reactant molecules are easily reduced.

In an exemplary aspect, the method for predicting reactant molecules and the method for training molecule completion models provided in the aspects of this disclosure may be applied to various scenarios, including, but not limited to, a cloud technology, artificial intelligence (AI), intelligent transportation, aided driving, and the like.

AI involves a theory, a method, a technology, and an application system that use a digital computer or a machine controlled by the digital computer to simulate, extend, and expand human intelligence, perceive an environment, obtain knowledge, and use knowledge to obtain an optimal result. In other words, AI, a comprehensive technology in computer science attempts to understand the essence of intelligence and produce a new intelligent machine that can respond in a manner similar to human intelligence. AI is to study the design principles and implementation methods of various intelligent machines, to enable the machines to have the functions of perception, reasoning, and decision making.

The AI technology is a comprehensive discipline, and relates to a wide range of fields including both hardware-level technologies and software-level technologies. The basic AI technologies generally include technologies such as a sensor, a dedicated AI chip, cloud computing, distributed storage, a big data processing technology, an operating/interaction system, and electromechanical integration. AI software technologies mainly include several major directions such as a computer vision technology, a speech processing technology, a natural language processing technology, machine learning/deep learning, automatic driving, and intelligent transportation.

The solutions provided in the aspects of this disclosure relate to a machine learning (ML) technology in the AI technologies. ML is a multi-field interdiscipline, and relates to a plurality of disciplines such as the probability theory, statistics, the approximation theory, convex analysis, and the algorithm complexity theory. ML specializes in studying how a computer simulates or implements a human learning behavior to obtain new knowledge or skills, and reorganize an existing knowledge structure, so as to keep improving its performance. ML is the core of AI, is a basic way to make the computer intelligent, and is applied to various fields of AI. ML and deep learning generally include technologies such as an artificial neural network, a belief network, reinforcement learning, transfer learning, inductive learning, and learning from instruction.

With the research and progress of the AI technology, the AI technology has been researched and applied in multiple fields, such as common smart homes, smart wearable devices, virtual assistants, smart speakers, intelligent marketing, unmanned driving, automatic driving, unmanned aerial vehicles, robots, smart healthcare, intelligent customer services, Internet of vehicles, and intelligent transportation. It is believed that with the development of the technology, the AI technology will be applied in more fields and can play increasingly important roles.

FIG. 1 shows a schematic diagram of an implementation environment provided in an aspect of this disclosure. The implementation environment may include: a terminal 11 and a server 12.

The method for predicting reactant molecules provided in the aspects of this disclosure may be performed by the terminal 11, the server 12, or the terminal 11 and the server 12 together, which is not limited in the aspect of this disclosure. In a case that the method for predicting reactant molecules provided in the aspects of this disclosure is performed by the terminal 11 and the server 12 together, the server 12 undertakes the main computing work, and the terminal 11 undertakes the secondary computing work; or, the server 12 undertakes the secondary computing work, and the terminal 11 undertakes the main computing work; or a distributed computing architecture is used between the server 12 and the terminal 11 for collaborative computing.

The method for training molecule completion models provided in the aspects of this disclosure may be performed by the terminal 11, the server 12, or the terminal 11 and the server 12 together, which is not limited in the aspect of this disclosure. In a case that the method for training molecule completion models provided in the aspects of this disclosure is performed by the terminal 11 and the server 12 together, the server 12 undertakes the main computing work, and the terminal 11 undertakes the secondary computing work; or, the server 12 undertakes the secondary computing work, and the terminal 11 undertakes the main computing work; or a distributed computing architecture is used between the server 12 and the terminal 11 for collaborative computing.

An execution device for the method for predicting reactant molecules and an execution device for the method for training molecule completion models may be the same or different, which is not limited in the aspect of this disclosure.

The terminal 11 may be any electronic product capable of interacting with users through one or more of a keyboard, a touchpad, a touch screen, a remote controller, a voice interaction device, a handwriting device and the like, such as a personal computer (PC), a mobile phone, a smart phone, a personal digital assistant (PDA), a wearable device, a pocket PC (PPC), a tablet computer, a smart car machine, a smart television, a smart speaker, a smart voice interaction device, a smart home appliance, an on board terminal, a virtual reality (VR) device, and an augmented reality (AR) device. The server 12 may be one server, a server cluster including a plurality of servers, or a cloud computing service center. A communication connection is established between the terminal 11 and the server 12 by a wired network or a wireless network.

Those skilled in the art understand that the foregoing terminal 11 and server 12 are only examples, and other existing or future potential terminals or servers applicable to this disclosure are also included within the protection scope of this disclosure and are included herein by reference.

The method for predicting reactant molecules provided in the aspect of this disclosure is used for predicting a reactant molecule for generating the product molecule according to the provided product molecule. The prediction task can be referred to as an inverse synthesis prediction task. The inverse synthesis prediction task is of great significance for the fields of chemistry and pharmaceuticals. Related inverse synthesis prediction tasks are mostly implemented based on synthesis reaction templates. For example, first, a template matched with a product molecule is found from synthesis reaction templates by a matching algorithm, and then, a reactant molecule is obtained according to the matched template. This type of method obtains certain effects in inverse synthesis prediction tasks, but the method based on a synthesis reaction template has two relatively significant defects: the first defect is that the method based on the synthesis reaction template is difficult to be generalized to a new reaction type, resulting in frequent updating of the synthesis reaction template, and summarizing the template requires a lot of work from chemical experts, resulting in very high cost; and the second defect is that the synthesis reaction template only summarizes some molecule-level reaction rules, but cannot grasp global correct information, often leading to incorrect prediction.

With the rise of the deep learning technology, in order to overcome the defects of the method based on the synthesis reaction template, deep learning models are widely applied to inverse synthesis prediction tasks. Reactant molecules of product molecules can be directly predicted by the deep learning technology without the need for matching with synthesis reaction templates. The deep learning technology can achieve strong inverse synthesis prediction effects, and the strong inverse synthesis prediction effects can help chemical experts discover possible synthesis paths of product molecules, thus greatly improving the research and development efficiency of new compounds. For example, product molecules may be drug molecules, so that the research and development efficiency of new drugs in the pharmaceutical industry can be greatly improved. In addition, the strong inverse synthesis prediction effects can also reveal some hidden scientific laws, provide new scientific knowledge, and discover new synthesis paths and even new synthesis reactions. The method for predicting reactant molecules provided in the aspect of this disclosure is a method for implementing inverse synthesis prediction tasks based on the deep learning technology.

Exemplarily, an inverse synthesis prediction task can be represented as (G^{P}→G^{R}), G^{P}=(V^{P}, B^{P}, A^{P}) represents a series of product molecules, and G^{R}=(V^{R}, B^{R}, A^{R}) represents a series of reactant molecules. V^{P} represents a set of atoms in the product molecule G^{P}, and a size of the set represents the number N (N is an integer not less than 1) of atoms in the product molecule G^{P}, that is, |V^{P}|=N.

B^{P}∈R^{N×N×C} represents the chemical bond link information of the product molecule G^{P}, the chemical bond link information represents the chemical bond link situation between atoms in the product molecule G^{P}, the chemical bond link information is a three-dimensional matrix, C (C is an integer not less than 1) represents the number of types of chemical bonds that may exist between atoms, and the value of the element [i, j, c] in the B^{P} represents whether the atom i and the atom j in the product molecule G^{P} are linked by a c-type chemical bond; in a case that the value of the [i, j, c] is 1, this represents that the atom i and the atom j in the product molecule G^{P} are linked by a c-type chemical bond; and in a case that the value of the [i, j, c] is 0, this represents that the atom i and the atom j in the product molecule G^{P} are not linked by a c-type chemical bond. Exemplarily, the chemical bond link information may also be referred to as an adjacency matrix.

A^{P}∈R^{N×F} represents the atomic feature information of the product molecule G^{P}, the atomic feature information includes the sub-feature information of each atom in the product molecule G^{P}, each atom has the sub-feature information with a dimension F (F is an integer not less than 1), and the obtaining manner of the sub-feature information of atoms will be introduced hereinafter and will not be described herein again. The meanings of the V^{R}, B^{R}, A^{R} can refer to the meanings of the V^{P}, B^{P}, A^{P}, and will not be described herein again.

The current inverse synthesis prediction task usually focuses on single product molecules and single-step inverse synthesis (multi-step inverse synthesis may be obtained by combining the results of single-step inverse synthesis), where the number of product molecules is 1, that is, |G^{P}|=1, and the number of reactant molecules is T (T is an integer not less than 1), that is, |G^{R}|=T, T≥1. The synthesis reaction follows the principle of atom-mapping, that is, atoms in the product molecule correspond to atoms in the reactant molecule one by one, so the product molecule and the reactant molecule share the same atom set. Because the inverse synthesis prediction task does not provide by-product molecules at one end of the product molecule, the number of atoms in the product molecule is usually less than the number of atoms in the reactant molecule, that is, |V^{R}|≥ |V^{P}|.

An aspect of this disclosure provides a method for predicting reactant molecules. The method can be applied to the foregoing implementation environment shown in FIG. 1. The method for predicting reactant molecules is performed by a computer device. The computer device may be the terminal 11 or the server 12, which is not limited in the aspect of this disclosure. As shown in FIG. 2, the method for predicting reactant molecules provided in the aspect of this disclosure may include step 201 to step 203.

Step 201: Obtain, by a computer device, a product molecule.

The product molecule refers to any compound molecule of a reactant molecule to be predicted. By predicting the reactant molecule of the product molecule, a synthesis path of the product molecule can be derived, thus providing data support for the research and development of product molecules. The aspect of this disclosure does not limit the type of the product molecule. For example, the product molecule may be a drug molecule, a clothing molecule, or a food molecule. The product molecule includes a plurality of atoms which are linked by chemical bonds. The types and number of the atoms included in the product molecule as well as the chemical bond link situation between the plurality of atoms are related to the product molecule, and are not limited in the aspect of this disclosure. Exemplarily, the product molecule may also be referred to as a resultant molecule.

The aspect of this disclosure does not limit the obtaining manner of the product molecule. Exemplarily, the product molecule may be extracted from a compound molecule database, or the product molecule may be selected from compound molecules published in journals or articles, or a compound molecule uploaded by a technician may be used as the product molecule.

The aspect of this disclosure does not limit the representation form of the product molecule, as long as the representation form can indicate the situation of atoms in the product molecule and the chemical bond link situation between atoms. Exemplarily, the representation form of the product molecule may be a name, a molecular formula, a character string, or the like. Exemplarily, the name of the product molecule can be obtained by naming the product molecule according to compound naming rules. The molecular formula of the product molecule is the most intuitive representation form of a composition structure of the product molecule, and the product molecule can be determined intuitively according to the molecular formula of the product molecule.

The character string of the product molecule is a character string generated according to certain specifications, which can represent the product molecule more concisely. Exemplarily, the specification for the character string for generating the product molecule may refer to a simplified molecular input line entry specification (SMILES). Exemplarily, in a case that the specification for the character string for generating the product molecule is the SMILES, the character string of the product molecule may also be referred to as an SMILES expression of the product molecule. The SMILES is a specification that explicitly describes a molecular structure using an American standard code for information interchange (ASCII) character string. Each compound molecule has a unique SMILES expression.

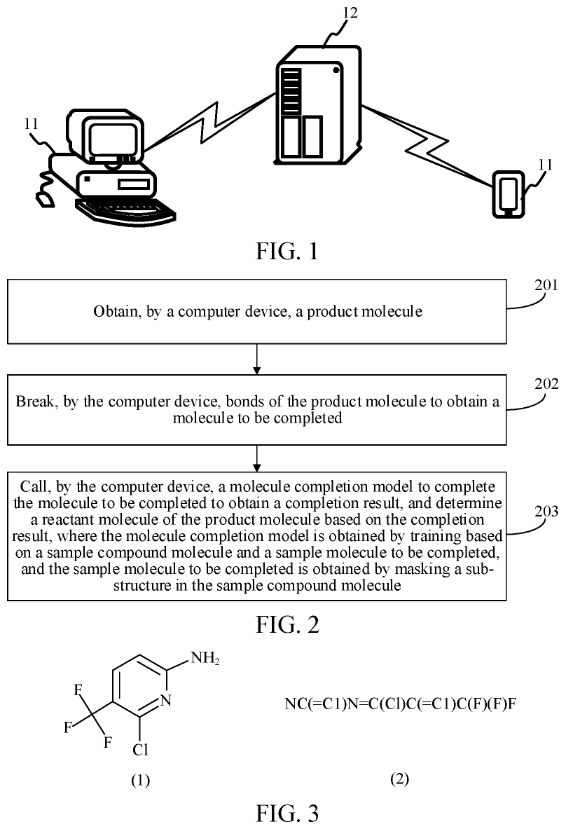

Exemplarily, the same product molecule may be represented by the molecular formula shown in FIG. 3 (1) or represented by the SMILES expression shown in FIG. 3 (2).

Step 202: Break, by the computer device, bonds of the product molecule to obtain a molecule to be completed. For example, bonds of the product molecule to be broken are selected to obtain a molecule to be completed, where the product molecule defines a compound molecule of a reactant molecule to be predicted. The molecule to be completed is a molecule obtained by breaking bonds of the product molecule. In some aspects, the molecule to be completed may also be referred to as a synthon. The molecule to be completed can be regarded as a molecule obtained by removing some molecular structures from the reactant molecule during the synthesis of the product molecule. After at least one molecule to be completed is determined, the molecule to be completed can be further completed to predict the reactant molecule. Exemplarily, the molecular structure removed from the reactant molecule can be referred to as a leaving group.

One or a plurality of molecules to be completed may be obtained based on the product molecule depending on the actual situation of the product molecule, which is not limited in the aspect of this disclosure.

In a possible implementation, the implementation process of breaking bonds of the product molecule by the computer device to obtain the molecule to be completed includes step 2021 to step 2023.

Step 2021: Obtain, by the computer device, graph structure information of the product molecule.

The graph structure information of the product molecule is information for representing a graph structure of the product molecule, and the graph structure of the product molecule may be a unique determined graph structure obtained by transforming the product molecule. Exemplarily, each atom in the product molecule is regarded as a node, and each chemical bond in the product molecule is regarded as an edge. From this perspective, the product molecule is transformed into a graph structure. That is to say, the graph structure of the product molecule is a graph structure constructed with atoms in the product molecule as nodes and chemical bonds in the product molecule as edges.

The aspect of this disclosure does not limit the type of the graph structure information of the product molecule, as long as the graph structure information can represent the graph structure of the product molecule. Exemplarily, the graph structure information of the product molecule includes atomic feature information of the product molecule and chemical bond link information of the product molecule. Exemplarily, the atomic feature information of the product molecule is used for representing the features of atoms in the product molecule. The chemical bond link information of the product molecule is used for representing the chemical bond link situation between atoms in the product molecule.

Because the graph structure of the product molecule takes the atoms in the product molecule as nodes and the chemical bonds in the product molecule as edges, the atomic feature information of the product molecule can be regarded as information for representing nodes in the graph structure of the product molecule, and the chemical bond link information of the product molecule can be regarded as information for representing edges in the graph structure of the product molecule. As a result, the atomic feature information of the product molecule and the chemical bond link information of the product molecule can be used for representing the graph structure of the product molecule. Then, the obtaining manner of the atomic feature information of the product molecule and the obtaining manner of the chemical bond link information of the product molecule are introduced respectively.

The atomic feature information of the product molecule includes sub-feature information of each atom in the product molecule, and the sub-feature information of each atom is used for representing the features of the atom. The aspect of this disclosure does not limit the representation form of the sub-feature information of atoms. For example, the representation form of the sub-feature information of atoms may be a matrix or a vector. The sub-feature information of different atoms has the same dimension, and the same dimension may be set according to experiences or flexibly adjusted according to application scenarios, which is not limited in the aspect of this disclosure. Exemplarily, in a case that the representation form of the sub-feature information of atoms is a matrix, the atomic feature information of the product molecule may also be referred to as an atomic feature matrix of the product molecule, and elements in each row in the atomic feature matrix represent the sub-feature information of an atom.

The principle of obtaining the sub-feature information of each atom is the same, and the aspect of this disclosure takes the manner of obtaining the sub-feature information of an atom as an example for description. Exemplarily, the manner of obtaining the sub-feature information of an atom includes the steps of: obtaining attribute information of the atom; and performing feature extraction on the attribute information of the atom to obtain the sub-feature information of the atom. The attribute information of the atom is used for describing the attributes of the atom, and the attribute information of the atom may be set according to experiences or flexibly adjusted according to application scenarios. Exemplarily, the attribute information of the atom includes but is not limited to at least one of the element information, valence state information and degree information of the atom, and information for indicating whether the atom belongs to a benzene ring.

The element information includes but is not limited to at least one of the ranking of the atom in the periodic table of elements, the symbol representation of the element, and the relative atomic mass. For example, the ranking of the carbon element in the periodic table of elements is sixth, the symbol representation of the carbon element is C, and the relative atomic mass of the carbon element is 12.01. The valence state information refers to a valence state of the atom in the product molecule, the valence state is also referred to as a chemical valence or an atomic valence, and the valence state refers to the number of chemical combination of an atom or an atomic group of various elements, a radical (root), and other atoms. The valence states of the atom in different compounds may be the same or different. For example, in CO (carbon monoxide), the valence state of carbon is +2 valence, while in CO₂ (carbon dioxide), the valence state of carbon is +4 valence. The degree information includes the number of other atoms linked to this atom. For CO₂, a carbon atom is linked to two oxygen atoms, and the two oxygen atoms are respectively linked to the carbon atom. Thus, the degree information of the carbon atom may be 2. The information for indicating whether the atom belongs to a benzene ring indicates whether the atom is an atom for forming a benzene ring.

After the attribute information of the atom is obtained, feature extraction is performed on the attribute information of the atom, and the information obtained by extraction is used as the sub-feature information of the atom. Exemplarily, the manner of performing feature extraction on the attribute information of the atom may be set according to experiences. For example, an atomic feature extraction model is called to perform feature extraction on the attribute information of the atom. Exemplarily, the atomic feature extraction model may be obtained in a manner of supervised training based on the attribute information of a sample atom and a feature label of the sample atom.

The chemical bond link information of the product molecule is determined based on the chemical bond link situation between atoms in the product molecule. Exemplarily, the chemical bond link information of the product molecule may also be referred to as an adjacency matrix of the graph structure of the product molecule. Exemplarily, the chemical bond link information of the product molecule is an N*N*C-dimensional matrix, where both N and C are integers not less than 1, N represents the number of atoms in the product molecule, and C represents the number of candidate chemical bond types. The candidate chemical bond types are set according to experiences or flexibly adjusted according to application scenarios, which are not limited in the aspect of this disclosure. Exemplarily, the candidate chemical bond types include common chemical bond types in synthesis reactions. For example, the candidate chemical bond types include but are not limited to a single bond, a double bond, a triple bond, an aromatic bond, an ionic bond, a covalent bond, and a metallic bond.

The value of the element [i, j, c] located at the i-th row, j-th column and a-th depth in the chemical bond link information of the product molecule represents whether the atom i and the atom j are linked by an a-type chemical bond, and the a-type chemical bond refers to the a-th candidate chemical bond type in C candidate chemical bond types, where both i and j are any values from 1 to N, and a is any value from 1 to C. In a case that the value of the [i, j, a] is 1, this represents that the atom i and the atom j are linked by the a-type chemical bond; and in a case that the value of the [i, j, a] is 0, this represents that the atom i and the atom j are not linked by the a-type chemical bond. The chemical bond link information of the product molecule can be obtained by analyzing the chemical bond link situation between atoms in the product molecule. The chemical bond link situation refers to whether atoms are linked by a chemical bond and what type of chemical bond is used when atoms are linked by a chemical bond.

In an exemplary aspect, the graph structure information of the product molecule further includes chemical bond link information of the product molecule. By additionally considering the chemical bond link information, more data support can be provided for the subsequent prediction process of the breakage probability of the chemical bond, thus improving the prediction accuracy of the breakage probability of the chemical bond. Exemplarily, the chemical bond link information includes sub-feature information of each chemical bond in the product molecule.

Exemplarily, the obtaining manner of the sub-feature information of the chemical bond includes the steps of: obtaining attribute information of the chemical bond, and performing feature extraction on the attribute information of the chemical bond to obtain the sub-feature information of the chemical bond. Exemplarily, the attribute information of the chemical bond is used for describing the attributes of the chemical bond, and the attribute information of the chemical bond may be set according to experiences or flexibly adjusted according to application scenarios, which is not limited in the aspect of this disclosure. Exemplarily, the attribute information of the chemical bond includes but is not limited to at least one of the bond type, conjugate feature, cyclic bond feature, bond energy, and bond distance of the chemical bond.

The bond type represents the type of the chemical bond, such as a single bond, a double bond, a triple bond, an aromatic bond, an ionic bond, a covalent bond, or a metallic bond. The conjugate feature represents whether the chemical bond is conjugated. The cyclic bond feature represents whether the chemical bond is a portion of a cyclic bond. The bond energy is a physical quantity for measuring the strength of the chemical bond based on energy factors. In general, the larger the bond energy, the firmer the chemical bond, and the less likely the chemical bond is to break. The bond distance refers to the shortest distance required for forming a chemical bond between two or more atomic nuclei.

After the attribute information of the chemical bond is obtained, feature extraction is performed on the attribute information of the chemical bond, and the information obtained by extraction is used as the sub-feature information of the chemical bond. Exemplarily, the manner of performing feature extraction on the attribute information of the chemical bond may be set according to experiences. For example, a chemical bond feature extraction model is called to perform feature extraction on the attribute information of the chemical bond. Exemplarily, the chemical bond feature extraction model may be obtained in a manner of supervised training based on the attribute information of a sample chemical bond and a feature label of the sample chemical bond.

Step 2022: Predict, by the computer device, breakage probabilities of chemical bonds in the product molecule based on the graph structure information, and determine the chemical bonds with the breakage probabilities meeting reference conditions as breakage chemical bonds in the product molecule.

The breakage probability of the chemical bond indicates the possibility that the chemical bond is a chemical bond formed in a synthesis reaction. The breakage probability of the chemical bond is positively correlated with the possibility that the chemical bond is a chemical bond formed in a synthesis reaction. That is, the higher the breakage probability of the chemical bond, the higher the possibility that the chemical bond is a chemical bond formed in a synthesis reaction. Exemplarily, the higher the possibility that the chemical bond is a chemical bond formed in a synthesis reaction, the higher the reliability of breaking bonds of the product molecule according to the chemical bond. The breakage probability of the chemical bond in the product molecule refers to the breakage probability of each chemical bond in the product molecule.

The breakage probability of the chemical bond may be predicted based on the graph structure information. The graph structure information of the product molecule can indicate the presence situation of chemical bonds in the product molecule, for example, which chemical bonds exist, and the situation of atoms linked to each chemical bond. The breakage probability of each chemical bond in the product molecule can be predicted according to the graph structure information. In an exemplary aspect, based on the graph structure information, the process of predicting the breakage probability of the chemical bond in the product molecule may be implemented by running a pre-written program, or implemented by calling a graph neural network model (or training graph neural network model).

The aspect of this disclosure calls the graph neural network model to predict the breakage probability of the chemical bond in the product molecule based on the graph structure information as an example for description. The graph neural network model is a model capable of processing the graph structure information of the compound molecule to predict the breakage probability of the chemical bond in the compound molecule, that is, a model capable of distinguishing which chemical bonds in the product molecule are more prone to breakage. The aspect of this disclosure does not limit the model structure of the graph neural network model. Exemplarily, the graph neural network model may be any graph-based deep learning network model, and the design of the graph neural network model may be simple or complex. For example, the graph neural network model may be a graph convolutional network (GCN) model, a graph attention network (GAT) model, a message passing neural network (MPNN) model, or the like.

The process of calling the graph neural network model to predict the breakage probability of the chemical bond in the product molecule based on the graph structure information is an internal processing process of the graph neural network model, and is related to a model structure of the graph neural network model, which is not limited in the aspect of this disclosure. Exemplarily, the process of calling the graph neural network model to predict the breakage probability of the chemical bond in the product molecule based on the graph structure information includes the steps of: calling the graph neural network model to extract target features of the chemical bond in the product molecule based on the graph structure information; and predicting the breakage probability of the chemical bond in the product molecule based on the target features of the chemical bond in the product molecule. The target features of the chemical bond are features for predicting the breakage probability of the chemical bond.

Exemplarily, the breakage probability of the chemical bond may be 1 or 0. In this case, the process of predicting the breakage probability of the chemical bond can be regarded as a process of binary prediction of the chemical bond. In a case that the breakage probability is 1, this represents that the chemical bond is very likely to be a chemical bond formed in a synthesis reaction; and in a case that the breakage probability is 0, this represents that the chemical bond is less likely to be a chemical bond formed in a synthesis reaction. Certainly, in an exemplary aspect, the breakage probability of the chemical bond may also be any probability between 0 and 1, which is not limited in the aspect of this disclosure.

After the breakage probabilities of the chemical bonds in the product molecule are determined, the chemical bonds with the breakage probabilities meeting reference conditions can be determined from the chemical bonds in the product molecule, and the chemical bonds with the breakage probabilities meeting reference conditions are determined as breakage chemical bonds in the product molecule. The breakage chemical bond is a chemical bond for obtaining the molecule to be completed from the product molecule. Exemplarily, the breakage chemical bond may also be referred to as a reaction site.

The chemical bond with the breakage probability meeting reference conditions refers to a chemical bond which is very likely to be formed in a synthesis reaction, that is, a chemical bond which is more prone to breakage. The process of breaking bonds of the product molecule according to this chemical bond has higher reliability, thus improving the reliability of the obtained molecule to be completed, and further improving the reliability of the predicted reactant molecule.

The breakage probability meeting reference conditions may be set according to experiences or flexibly adjusted according to application scenarios, which is not limited in the aspect of this disclosure. In an exemplary aspect, the breakage probability meeting reference conditions means that the breakage probability is not less than a probability threshold. The probability threshold may be set according to experiences or flexibly adjusted according to application scenarios. For example, the probability threshold is 0.5, or the probability threshold is 0.8. In an exemplary aspect, the breakage probability meeting reference conditions may also mean that breakage probabilities are first L (L is an integer not less than 1) large breakage probabilities in the breakage probabilities of all chemical bonds. The value of L may be set according to experiences or flexibly adjusted according to application scenarios. For example, the value of L is 3, or the value of L is 2.

In an exemplary aspect, before calling the graph neural network model to predict the breakage probability of the chemical bond in the product molecule based on the graph structure information, the graph neural network model needs to be trained first. In an exemplary aspect, the process of training the graph neural network model includes the steps of: obtaining graph structure information of a training compound molecule and a standard breakage probability of chemical bonds in the training compound molecule; calling the graph neural network model to predict a training breakage probability of the chemical bonds in the training compound molecule based on the graph structure information of the training compound molecule; determining reference loss based on the difference between the standard breakage probability and the training breakage probability; updating model parameters of the graph neural network model based on the reference loss; and determining the model obtained by current training as a graph neural network model after training in response to the training process meeting a first termination condition.

Meeting the first termination condition may be set according to experiences or flexibly adjusted according to application scenarios. For example, meeting the first termination condition means that the reference loss converges, the reference loss is less than a first loss threshold, or the number of updates of the model parameters reaches a first number threshold. The first loss threshold and the first number threshold may be set according to experiences or flexibly adjusted according to application scenarios, which are not limited in the aspect of this disclosure.

The training compound molecule refers to a compound molecule capable of obtaining the graph structure information and the standard breakage probability of the chemical bond. There may be one or more training compound molecules, which is not limited in the aspect of this disclosure. Exemplarily, the principle of obtaining the graph structure information of the training compound molecule is the same as the principle of obtaining the graph structure information of the product molecule, and will not be described herein again. Exemplarily, the graph structure information of the training compound molecule can be stored in a database corresponding to the training compound molecule, so that the graph structure information of the training compound molecule can be directly extracted from the database.

The standard breakage probability of the chemical bond in the training compound molecule is a true breakage probability of the chemical bond in the training compound molecule, which is used for providing supervision information for the training process of the graph neural network model. Exemplarily, the standard breakage probability of the chemical bond in the training compound molecule may also be referred to as a breakage probability (ground-truth) label of the chemical bond in the training compound molecule. In an exemplary aspect, the standard breakage probability of the chemical bond in the training compound molecule is stored in the database corresponding to the training compound molecule, so that the standard breakage probability of the chemical bond in the training compound molecule can be directly extracted from the database.

In an exemplary aspect, the training compound molecule is a molecule synthesized by a known synthesis reaction. In this case, the standard breakage probability of the chemical bond in the training compound molecule can be obtained by comparing the training compound molecule with the reactant molecule for synthesizing the training compound molecule. Exemplarily, the process of obtaining the standard breakage probability of the chemical bond in the training compound molecule by comparing the training compound molecule with the reactant molecule for synthesizing the training compound molecule may be as follows: in a case that an atom u and an atom v in the product molecule are linked by an a-type chemical bond bᵤᵥ (which can be represented as B″[u, v, a]=1, for a certain a), and the atom u and the atom v in the reactant molecule for synthesizing the training compound molecule are not linked by any type of chemical bond (which can be represented as B^{R}[u, v, a]=0, for any a), the standard breakage probability of the chemical bond bᵤᵥ between the atom u and the atom v in the target product molecule is denoted as 1 (which can be represented as yᵤᵥ=1); and in a case that the atom u and the atom v in the reactant molecule for synthesizing the training compound molecule are linked by a certain type of chemical bond, the standard breakage probability of the chemical bond bᵤᵥ between the atom u and the atom v in the target product molecule is denoted as 0 (which can be represented as yᵤᵥ=0).

The implementation principle of calling the graph neural network model to predict the training breakage probability of the chemical bond in the training compound molecule based on the graph structure information of the training compound molecule is the same as the implementation principle of calling the graph neural network model to predict the breakage probability of the chemical bond in the product molecule based on the graph structure information of the product molecule, and will not be described herein again.

After obtaining the training breakage probability of the chemical bond in the training compound molecule, the reference loss is determined based on the difference between the standard breakage probability and the training breakage probability. The aspect of this disclosure does not limit the measuring manner of the difference between the standard breakage probability and the training breakage probability. Exemplarily, the difference between the standard breakage probability and the training breakage probability refers to the cross entropy difference between the standard breakage probability and the training breakage probability, or the difference between the standard breakage probability and the training breakage probability refers to the mean square difference between the standard breakage probability and the training breakage probability.

Exemplarily, in a case that the difference between the standard breakage probability and the training breakage probability refers to the cross entropy difference between the standard breakage probability and the training breakage probability, the reference loss can be calculated based on Formula 1:

$$L = - \frac{1}{K} {\sum }_{k = 1}^{K} {\sum }_{( {b}_{u v} ∈ {B}_{k}^{P} )} {b}_{u v} ( {y}_{u v} log {\tilde{s}}_{u v} + ( 1 - {y}_{u v} ) log ( 1 - {\tilde{s}}_{u v} ) ) \qquad ( Formula 1 )$$

where L represents the reference loss; K (K is an integer not less than 1) represents the number of training compound molecules; the Bₖ^{P} represents the chemical bond link information of the k-th (the value of k ranges from 1 to K) training compound molecule; the bᵤᵥ ∈Bₖ^{P} represents the chemical bond in the k-th training compound molecule determined according to B^{R}, and the reference loss is obtained by comprehensively considering each chemical bond in each training compound molecule; the yᵤᵥ represents the standard breakage probability of the chemical bond bᵤᵥ; and the s̅ᵤᵥ represents the training breakage probability of the chemical bond bᵤᵥ.

Step 2023: Break, by the computer device, bonds of the product molecule based on the breakage chemical bonds to obtain the molecule to be completed.

Exemplarily, breaking bonds of the product molecule based on the breakage chemical bonds refers to breaking the breakage chemical bonds in the product molecule, and each molecule obtained after breaking the breakage chemical bonds in the product molecule is used as each molecule to be completed. One or more molecules to be completed may be obtained by breaking bonds of the product molecule, which is not limited in the aspect of this disclosure.

Exemplarily, the molecule to be completed is represented as {Gₕ^{S}}ₕ₌₁^{H}, where H (H is an integer not less than 1) represents the number of molecules to be completed, and Gₕ^{S} represents the h-th (h is any value from 1 to H) molecule to be completed. Exemplarily, the process of obtaining the molecule to be completed on the basis of the product molecule (represented as G^{P}) can be regarded as a process of modeling probability distribution P({Gₕ^{S}}ₕ₌₁^{H}|G^{P}).

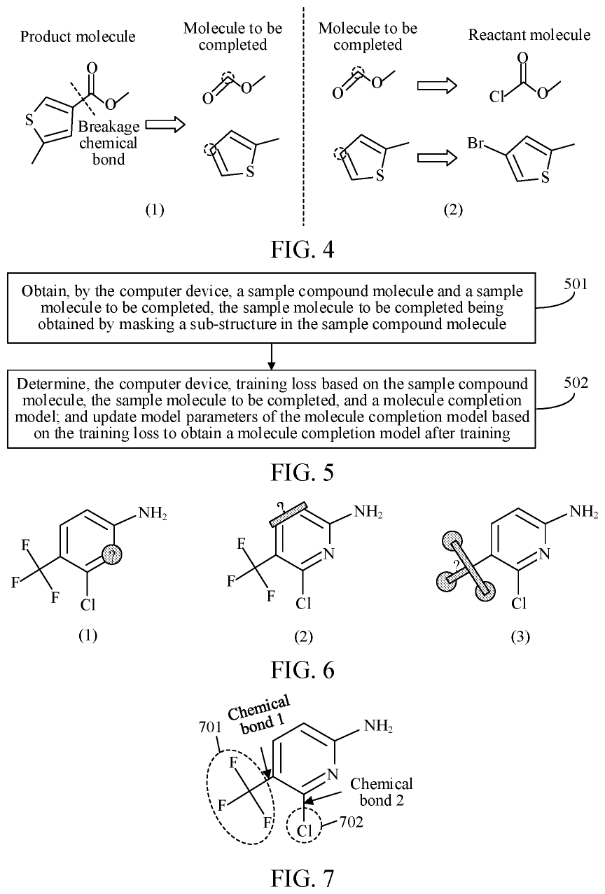

Exemplarily, the process of determining the molecule to be completed from the product molecule can be referred to as a reaction site prediction process, and the reaction site prediction process can be regarded as a first stage in a reactant molecule prediction process. Exemplarily, the first stage may be shown in FIG. 4 (1), there is one breakage chemical bond in the product molecule, and two molecules to be completed can be obtained after breaking the breakage chemical bond. The dashed circles on the two molecules to be completed in FIG. 4 (1) mark two atoms linked by the breakage chemical bond.

The process of breaking bonds of the product molecule based on step 2021 to step 2023 is only an exemplary implementation process, which is not limited in the aspect of this disclosure. In some aspects, breakage chemical bonds may also be selected from chemical bonds of the product molecule according to experiences, and then, the bonds of the product molecule are broken based on the selected breakage chemical bonds.

In step 203, the computer device calls a molecule completion model to complete the molecule to be completed to obtain a completion result, and determine a reactant molecule of the product molecule based on the completion result, where the molecule completion model is obtained by training based on a sample compound molecule and a sample molecule to be completed, and the sample molecule to be completed is obtained by masking a sub-structure in the sample compound molecule. For example, a molecule completion model is applied to complete the molecule to be completed to obtain a completion result indicating a reactant molecule of the product molecule based on the molecule to be completed. The molecule completion model is obtained, for example, by training based on sample compound molecules and sample molecules to be completed obtained by masking sub-structures in the sample compound molecules.

Exemplarily, masking the sub-structure in the sample compound molecule refers to hiding the sub-structure in the sample compound molecule. After the sub-structure in the sample compound molecule is masked, the original state of the sub-structure in the sample compound molecule cannot be obtained. The manner of masking the sub-structure in the sample compound molecule may be set according to experiences or flexibly adjusted according to actual application scenarios, as long as the sub-structure in the sample compound molecule can be hidden. Exemplarily, the manner of masking the sub-structure in the sample compound molecule may be implemented by replacing the sub-structure in the sample compound molecule with a specific structure, or by covering the sub-structure in the sample compound molecule. Exemplarily, the specific structure refers to a structure that is different from a real structure of a compound molecule, so as to distinguish a masked part from an unmasked part.

The process of predicting the reactant molecule on the basis of the molecule to be completed is implemented by calling the molecule completion model. The molecule completion model is obtained by training based on the sample compound molecule and the sample molecule to be completed obtained by masking the sub-structure in the sample compound molecule. The sample molecule to be completed is obtained on the basis of the sample compound molecule. The process of obtaining the molecule completion model by training based on the sample compound molecule and the sample molecule to be completed can be regarded as a process of masking the sample compound molecule and training the model to reconstruct the masked part. This training process is a process of training the model by a self-supervised learning strategy. In the process of training the model by the self-supervised learning strategy, a self-supervised learning task is a task designed under the concept of “Mask and Fill”. The basic idea is to mask a portion of the sub-structure of the compound molecule and then train a molecule completion model to reconstruct the sub-structure.

In the process of training by the self-supervised learning strategy, the data for model training is the data obtained on the basis of the sample compound molecule, and regardless of whether the sample compound is a compound in a known synthesis reaction, the data can be used as the data for model training. That is to say, the training process of the model is not limited by the known synthesis reaction. The molecule completion model trained by the training process has a stronger generalization ability, which is beneficial for effectively adapting to prediction scenarios of reactant molecules related to various synthesis reactions to expand adaptation scenarios, thus improving the prediction reliability and prediction accuracy of the reactant molecule.

In the aspect of this disclosure, it is assumed that the number of molecules to be completed is the same as the number of reactant molecules, that is, one molecule to be completed corresponds to one reactant molecule. Calling the molecule completion model to complete the molecule to be completed refers to calling the molecule completion model to respectively complete each molecule to be completed. After completion, each molecule to be completed corresponds to a completion result, and a reactant molecule of the product molecule can be determined according to the completion result of each molecule to be completed.

The principle of calling the molecule completion model to complete each molecule to be completed is the same. The aspect of this disclosure takes the process of calling the molecule completion model to complete the molecule to be completed as an example for description. In a case that there are multiple molecules to be completed, the molecule completion model can be called to complete the multiple molecules to be completed in parallel, so as to improve the efficiency of predicting the reactant molecule.

Calling the molecule completion model to complete the molecule to be completed can obtain the completion result of the molecule to be completed. The aspect of this disclosure does not limit the form of the completion result of the molecule to be completed, as long as the reactant molecule can be determined according to the completion result of the molecule to be completed. The completion result indicates a molecule after completion.

Exemplarily, the completion result of the molecule to be completed may be graph structure information of the molecule after completion of the molecule to be completed. In this case, the molecule after completion of the molecule to be completed can be determined according to the graph structure information, and the molecule after completion of the molecule to be completed is used as a reactant molecule.

Exemplarily, the completion result of the molecule to be completed may also be indication information of a missing structure in the molecule to be completed. The indication information includes information for representing the missing structure in the molecule to be completed and information for indicating the position in the molecule to be completed to be linked with the missing structure. In this case, the missing structure can be determined according to the indication information, then, the missing structure is linked to the molecule to be completed, and the molecule obtained by linking is used as a reactant molecule. Exemplarily, the information for representing the missing structure in the molecule to be completed may be the graph structure information, molecular formula, molecular character string, or the like of the missing structure in the molecule to be completed, which is not limited in the aspect of this disclosure.

Exemplarily, in a case that the missing structure in the molecule to be completed is a single atom or single chemical bond level structure, the completion result of the molecule to be completed may also be a classification result and a link position prediction result of the missing structure in the molecule to be completed. The classification result includes a matching probability of the missing structure in the molecule to be completed and a reference structure (such as a reference atom or a reference chemical bond). The link position prediction result indicates the position in the molecule to be completed to be linked with the missing structure. In this case, the type of the missing structure in the molecule to be completed (that is, the type of a single atom or a single chemical bond) can be determined according to the classification result, then, the structure of this type is linked to the molecule to be completed at the position in the molecule to be completed to be linked with the missing structure, and the molecule obtained by linking is used as a reactant molecule.

The process of calling the molecule completion model to complete the molecule to be completed to obtain the completion result of the molecule to be completed is an internal processing process of the molecule completion model, and is related to a structure of the molecule completion model, which is not limited in the aspect of this disclosure.

In an exemplary aspect, the molecule completion model is a flow-based generative model, and the flow-based generative model is an invertible model. The flow-based generative model is introduced below.

The flow-based generative model directly performs a maximum likelihood estimation. Furthermore, the flow-based generative model can provide a likelihood estimation of a generated result, that is, the flow-based generative model has stronger interpretability. The general idea of the flow-based generative model is to transform complex data probability distribution into common simple distribution (also referred to as hidden variable distribution) through a series of invertible transformations. Assuming that the data is derived from distribution X˜P_{X}(X), the hidden variable distribution is Z˜P_{Z}(Z) (generally Gaussian distribution), Z=f_{θ}(X) through a series of invertible function transformations f_{θ}=f_{L}∘f_{L-1} ∘ . . . ∘f₁, and the entire process is

$$x {↔}^{{f}_{1}} {Z}_{1} {↔}^{{f}_{2}} {Z}_{2} … {↔}^{{f}_{L}} {Z}_{L} ,$$

where L is an integer not less than 1. The target of the flow-based generative model is a maximum log-likelihood estimation

$${max}_{θ} log {p}_{θ} ( x ) ,$$

and an expression of a log-likelihood estimation log p_{θ}(x) can be derived from invertible mapping of the flow-based generative model, as shown in Formula 2:

$$log {p}_{θ} ( x ) = log {p}_{θ} ( z ) + log \vert det ( \frac{dz}{dx} ) \vert = log {p}_{θ} ( z ) + {\sum }_{i = 1}^{L} log \vert det ( \frac{d {z}_{i}}{d {z}_{i - 1}} ) \vert \qquad ( Formula 2 )$$

where the det(⋅) represents determinant calculation of a matrix, the dzᵢ/dzᵢ₋₁ is a n×n matrix in a high-dimensional case, which is referred to as a Jacobian matrix, the n represents a dimension of z, and n is an integer not less than 1; the p_{θ}(z) represents a probability of z under a parameter θ; and the p_{θ}(x) represents a probability of x under the parameter θ.

The flow-based generative model usually adopts a network layer design solution of a coupling layer to balance the calculation efficiency and the model representation ability. The relationship between input x and output z of the coupling layer is shown in Formula 3 and Formula 4:

$${z}_{1 : d} = {x}_{1 : d} \qquad ( Formula 3 )$$

$${z}_{d + 1 : n} = {x}_{d + 1 : n} ⊙ {e}^{{S}_{θ} ( {x}_{1 : d} )} + {T}_{θ} ( {x}_{1 : d} ) \qquad ( Formula 4 )$$

where Formula 3 represents copying of the first d (d is an integer not less than 1 and not greater than n) dimensions of the input x, and Formula 4 represents transformation of the remaining dimensions (from the (d+1)-th dimension to the n-th dimension) of the input x. The S_{θ}(⋅) and the T_{θ}(⋅) in Formula 4 represent two transformation functions, and the two transformation functions are used for outputting transformation information with the same dimension as x_{d+1:n}. Exemplarily, the S_{θ}(⋅) represents a scale function, and the Tθ(⋅) represents a transformation function. The ⊙ represents multiplication of elements at corresponding positions in a matrix. This design allows for an inverse operation of the coupling layer by simply transforming Formula 3 and Formula 4, thus achieving inverse transformations from z to x. The inverse operation is implemented based on Formula 5 and Formula 6 as follows:

$${x}_{1 : d} = {z}_{1 : d} \qquad ( Formula 5 )$$

$${x}_{d + 1 : n} = ( {z}_{d + 1 : n} - {T}_{θ} ( {z}_{1 : d} ) ) / {e}^{{S}_{θ} ( {z}_{1 : d} )} \qquad ( Formula 6 )$$

The design of the coupling layer transforms the Jacobian matrix into a diagonal matrix, and the determinant calculation of the diagonal matrix is a product of diagonal elements, that is,

$$det ( \frac{d z}{d x} ) = exp ( {\sum }_{j} {( {S}_{θ} ( {z}_{1 : d} ) )}_{j} ) ,$$

where j represents any element in S_{θ}(z_{1:d}). In this case, the scale function and the transformation function may be any complex neural network without increasing the calculation complexity of the determinant of the Jacobian matrix, thus improving the calculation efficiency.

In an exemplary aspect, in a case that the molecule completion model is a flow-based generative model, the process of calling the molecule completion model to complete the molecule to be completed to obtain the completion result of the molecule to be completed includes step 2031 to step 2034.

Step 2031: Determine, by the computer device, a target atomic feature hidden variable based on atomic feature information of the molecule to be completed, the target atomic feature hidden variable being an atomic feature hidden variable of the molecule after completion; and determine a target chemical bond link hidden variable based on chemical bond link information of the molecule to be completed, the target chemical bond link hidden variable being a chemical bond link hidden variable of the molecule after completion.

The atomic feature information of the molecule to be completed is used for representing the features of atoms in the molecule to be completed, and the target atomic feature hidden variable is used for making assumptions about the features of atoms in the molecule after completion of the molecule to be completed. The principle of obtaining the atomic feature information of the molecule to be completed is the same as the principle of obtaining the atomic feature information of the product molecule, and will not be described herein again.

In an exemplary aspect, the process of obtaining the target atomic feature hidden variable based on the atomic feature information of the molecule to be completed includes the steps of: sampling an atomic feature hidden variable of the missing structure of the molecule to be completed from the known probability distribution; and determining the target atomic feature hidden variable based on the atomic feature information of the molecule to be completed and the atomic feature hidden variable of the missing structure.

The missing structure of the molecule to be completed refers to a structure to be completed of the molecule to be completed, and the atomic feature hidden variable of the missing structure is used for making assumptions about the features of atoms in the missing structure. The atomic feature hidden variable of the missing structure is sampled from the known probability distribution, that is, the atomic feature hidden variable of the missing structure is a variable following the known probability distribution. The known probability distribution is any distribution capable of determining the probability of a variable following the probability distribution. The type of the known probability distribution may be set according to experiences, which is not limited in the aspect of this disclosure. For example, the known probability distribution may refer to Gaussian distribution or uniform distribution. Exemplarily, the manner of sampling the atomic feature hidden variable of the missing structure may be random sampling.

In an exemplary aspect, both the atomic feature hidden variable of the missing structure and the atomic feature information of the molecule to be completed are in the form of a matrix. In the matrix of the atomic feature hidden variable of the missing structure and the matrix of the atomic feature information of the molecule to be completed, a row of elements correspond to one atom. Exemplarily, the manner of determining the target atomic feature hidden variable based on the atomic feature information of the molecule to be completed and the atomic feature hidden variable of the missing structure may be as follows: the atomic feature information of the molecule to be completed and the atomic feature hidden variable of the missing structure are longitudinally spliced, so as to obtain the target atomic feature hidden variable based on the matrix obtained by splicing.

Exemplarily, the manner of obtaining the target atomic feature hidden variable based on the matrix obtained by splicing may be as follows: the matrix obtained by splicing is used as the target atomic feature hidden variable.

Exemplarily, the manner of obtaining the target atomic feature hidden variable based on the matrix obtained by splicing may also be as follows: in a case that the dimension of the matrix obtained by splicing is a first reference dimension, the matrix obtained by splicing is used as the target atomic feature hidden variable; and in a case that the dimension of the matrix obtained by splicing is less than the first reference dimension, the matrix obtained by splicing is expanded into a matrix with the first reference dimension, and the matrix obtained by expanding is used as the target atomic feature hidden variable. This manner can ensure that the dimension of the target atomic feature hidden variable is the first reference dimension, thus improving the normalization of the target atomic feature hidden variable.

The first reference dimension is a preset dimension parameter for constraining the information about atomic features. The first reference dimension can be considered as a dimension of the atomic feature information of a maximum reactant molecule, that is, the aspect of this disclosure considers that the first reference dimension is not less than the dimension of the matrix obtained by splicing. Exemplarily, the process of expanding the matrix obtained by splicing into the matrix with the first reference dimension may mean that: the matrix obtained by splicing is placed at an upper left corner, and 0 elements are added to other positions until the matrix with the first reference dimension is obtained.

The chemical bond link information of the molecule to be completed is used for representing the chemical bond link situation between atoms in the molecule to be completed, and the target chemical bond link hidden variable is used for making assumptions about the chemical bond link situation between atoms in the molecule after completion of the molecule to be completed. The principle of obtaining the chemical bond link information of the molecule to be completed is the same as the principle of obtaining the chemical bond link information of the product molecule, and will not be described herein again.

In an exemplary aspect, the process of determining the target chemical bond link hidden variable based on the chemical bond link information of the molecule to be completed includes the steps of: sampling a chemical bond link hidden variable of the missing structure of the molecule to be completed from the known probability distribution; and obtaining the target chemical bond link hidden variable based on the chemical bond link information of the molecule to be completed and the chemical bond link hidden variable of the missing structure.

The chemical bond link hidden variable of the missing structure is used for making assumptions about the chemical bond link situation between atoms in the missing structure. The chemical bond link hidden variable of the missing structure is sampled from the known probability distribution, that is, the chemical bond link hidden variable of the missing structure is a variable following the known probability distribution. Exemplarily, the manner of sampling the chemical bond link hidden variable of the missing structure may be random sampling.

Exemplarily, the chemical bond link hidden variable of the missing structure can indicate the chemical bond link situation (assumed situation) between atoms in the missing structure, and the chemical bond link information of the molecule to be completed can indicate the chemical bond link situation (real situation) between atoms in the molecule to be completed. Based on the chemical bond link hidden variable of the missing structure and the chemical bond link information of the molecule to be completed, target information for indicating the chemical bond link situation (assumed situation) between atoms in the missing structure and atoms in the molecule to be completed (that is, atoms in the molecule after completion) can be obtained, and the target chemical bond link hidden variable can be obtained based on the target information. Exemplarily, both the target information and the target chemical bond link hidden variable are in the form of a matrix.

The chemical bond link situation between atoms in the molecule after completion includes not only the chemical bond link situation between atoms in the missing structure and the chemical bond link situation between atoms in the molecule to be completed, but also the chemical bond link situation between atoms in a missing result and atoms in a completed molecule. The chemical bond link situation between atoms in the missing result and atoms in the completed molecule may be set according to experiences. For example, atoms in the default missing result and atoms in the completed molecule are not linked by chemical bonds.

Exemplarily, the manner of obtaining the target chemical bond link hidden variable based on the target information may be as follows: the target information is used as the target chemical bond link hidden variable.

Exemplarily, the manner of obtaining the target chemical bond link hidden variable based on the target information may also be as follows: in a case that the dimension of the target information is a second reference dimension, the target information is used as the target chemical bond link hidden variable; and in a case that the dimension of the target information is less than the second reference dimension, the target information is expanded into a matrix with the second reference dimension, and the matrix obtained by expanding is used as the target chemical bond link hidden variable. This manner can ensure that the dimension of the target chemical bond link hidden variable is the second reference dimension, thus improving the normalization of the target chemical bond link hidden variable.

The second reference dimension is a preset dimension parameter for constraining the information about chemical bond link. The second reference dimension can be considered as a dimension of the chemical bond link information of a maximum reactant molecule, that is, the aspect of this disclosure considers that the second reference dimension is not less than the dimension of the target information. Exemplarily, the process of expanding the target information into the matrix with the second reference dimension may mean that: the target information is placed at an upper left corner, and 0 elements are added to other positions until the matrix with the second reference dimension is obtained.

Step 2032: Call, by the computer device, the molecule completion model to transform the target chemical bond link hidden variable to obtain target chemical bond link information.

The target chemical bond link information is used for representing the chemical bond link situation between atoms in the molecule after completion of the molecule to be completed. The process of transforming the target chemical bond link hidden variable to obtain the target chemical bond link information refers to a process of predicting representation information of the chemical bond link situation between atoms in the molecule after completion of the molecule to be completed according to the assumed information of the chemical bond link situation between atoms in the molecule after completion of the molecule to be completed.

Exemplarily, the implementation process of calling the molecule completion model to transform the target chemical bond link hidden variable to obtain the target chemical bond link information includes the steps of: calling the molecule completion model to obtain the reference chemical bond link information of the molecule to be completed and the reference chemical bond link hidden variable of the missing structure based on the target chemical bond link hidden variable; transforming the reference chemical bond link hidden variable of the missing structure based on the reference chemical bond link information of the molecule to be completed to obtain the reference chemical bond link information of the missing structure; and obtaining the target chemical bond link information based on the reference chemical bond link information of the molecule to be completed and the reference chemical bond link information of the missing structure.

In an exemplary aspect, the target chemical bond link hidden variable is a matrix with the second reference dimension. The manner of obtaining the reference chemical bond link information of the molecule to be completed based on the target chemical bond link hidden variable is as follows: the information for indicating the chemical bond link situation between atoms in the molecule to be completed in the target chemical bond link hidden variable remains unchanged, and other information is set to 0 to obtain the reference chemical bond link information of the molecule to be completed. The reference chemical bond link information of the molecule to be completed obtained in this manner is also a matrix with the second reference dimension.

In an exemplary aspect, the manner of obtaining the reference chemical bond link hidden variable of the missing structure based on the target chemical bond link hidden variable is as follows: the information for indicating the chemical bond link situation between atoms in the missing structure in the target chemical bond link hidden variable remains unchanged, and other information is set to 0 to obtain the reference chemical bond link hidden variable of the missing structure. The reference chemical bond link hidden variable of the missing structure obtained in this manner is also a matrix with the second reference dimension.

Exemplarily, the implementation manner of transforming the reference chemical bond link hidden variable of the missing structure based on the reference chemical bond link information of the molecule to be completed to obtain the reference chemical bond link information of the missing structure may be as follows: first reference transformation information is obtained based on the reference chemical bond link information of the molecule to be completed; and the reference chemical bond link hidden variable of the missing structure is transformed through the first reference transformation information to obtain the reference chemical bond link information of the missing structure. Exemplarily, the first reference transformation information may be obtained based on at least one transformation function (such as a S_{θ}(⋅) transformation function or a T_{θ}(⋅) transformation function involved in Formula 6).

Exemplarily, the implementation process of transforming the reference chemical bond link hidden variable of the missing structure through the first reference transformation information to obtain the reference chemical bond link information of the missing structure can be represented by Formula 6, where the x_{d+1:n} is used for representing the reference chemical bond link information of the missing structure, the T_{θ}(z_{1:d}) and the S_{θ}(z_{1:d}) are used for representing the first reference transformation information obtained based on the z_{1:d}, the z_{1:d} is used for representing the reference chemical bond link information of the molecule to be completed, and the z_{d+1:n} is used for representing the reference chemical bond link hidden variable of the missing structure. The transformation does not change the dimension of the information, that is, the dimension of the reference chemical bond link hidden variable of the missing structure is the same as the dimension of the reference chemical bond link information of the missing structure.

Exemplarily, both the reference chemical bond link information of the molecule to be completed and the reference chemical bond link information of the missing structure are matrices with the second reference dimension. The manner of obtaining the target chemical bond link information based on the reference chemical bond link information of the molecule to be completed and the reference chemical bond link information of the missing structure may be as follows: elements at corresponding positions in the reference chemical bond link information of the molecule to be completed and the reference chemical bond link information of the missing structure are added, and the matrix obtained by adding is used as the target chemical bond link information. Exemplarily, a Cartesian product between the reference chemical bond link information of the molecule to be completed and the reference chemical bond link information of the missing structure may also be used as the target chemical bond link information.

Exemplarily, the molecule completion model includes a chemical bond completion model. Step 2032 can be implemented by calling the chemical bond completion model in the molecule completion model, that is, the chemical bond completion model in the molecule completion model is called to transform the target chemical bond link hidden variable to obtain the target chemical bond link information.

The chemical bond completion model can transform the chemical bond link hidden variable of a molecule into the chemical bond link information of the molecule, where the chemical bond link hidden variable of the molecule is used for making assumptions about the chemical bond link situation between atoms in the molecule, and the chemical bond link information of the molecule is used for representing the chemical bond link situation between atoms in the molecule. That is to say, the input of a target chemical bond completion model is assumed information of the chemical bond link situation between atoms in the molecule, and the output of the target chemical bond completion model is representation information of the chemical bond link situation between atoms in the molecule.

Exemplarily, the chemical bond completion model is an invertible model, that is, there is an inverse model of the chemical bond completion model. The inverse model of the chemical bond completion model can inversely transform the chemical bond link information of a molecule into the chemical bond link hidden variable of the molecule. That is to say, the input of the inverse model of the chemical bond completion model is representation information of the chemical bond link situation between atoms in the molecule, and the output of the inverse model of the chemical bond completion model is assumed information of the chemical bond link situation between atoms in the molecule.

The chemical bond completion model is obtained by training, and the model structure of the chemical bond completion model remains unchanged during the training process. The model structure of the chemical bond completion model can refer to the model structure introduced in the aspect shown in FIG. 5, and will not be described herein again.

Step 2033: Call, by the computer device, the molecule completion model to transform the target atomic feature hidden variable to obtain target atomic feature information.

The target atomic feature information is used for representing the features of atoms in the molecule after completion of the molecule to be completed. The process of transforming the target atomic feature hidden variable to obtain the target atomic feature information refers to a process of predicting representation information of the features of atoms in the molecule after completion of the molecule to be completed according to the assumed information of the features of atoms in the molecule after completion of the molecule to be completed.

Exemplarily, the implementation process of calling the molecule completion model to transform the target atomic feature hidden variable to obtain the target atomic feature information includes the steps of: calling the molecule completion model to obtain the reference atomic feature information of the molecule to be completed and the reference atomic feature hidden variable of the missing structure based on the target atomic feature hidden variable; transforming the reference atomic feature hidden variable of the missing structure based on the reference atomic feature information of the molecule to be completed to obtain the reference atomic feature information of the missing structure; and obtaining the target atomic feature information based on the reference atomic feature information of the molecule to be completed and the reference atomic feature information of the missing structure.

In an exemplary aspect, the target atomic feature hidden variable is a matrix with the first reference dimension. The manner of obtaining the reference atomic feature information of the molecule to be completed based on the target atomic feature hidden variable is as follows: the information for indicating the features of atoms in the molecule to be completed in the target atomic feature hidden variable remains unchanged, and other information is set to 0 to obtain the reference atomic feature information of the molecule to be completed. The reference atomic feature information of the molecule to be completed obtained in this manner is also a matrix with the first reference dimension.

In an exemplary aspect, the manner of obtaining the reference atomic feature hidden variable of the missing structure based on the target atomic feature hidden variable is as follows: the information for indicating the features of atoms in the missing structure in the target atomic feature hidden variable remains unchanged, and other information is set to 0 to obtain the reference atomic feature hidden variable of the missing structure. The reference atomic feature hidden variable of the missing structure obtained in this manner is also a matrix with the first reference dimension.

Exemplarily, the implementation manner of transforming the reference atomic feature hidden variable of the missing structure based on the reference atomic feature information of the molecule to be completed to obtain the reference atomic feature information of the missing structure may be as follows: second reference transformation information is obtained based on the reference atomic feature information of the molecule to be completed; and the reference atomic feature hidden variable of the missing structure is transformed through the second reference transformation information to obtain the reference atomic feature information of the missing structure. Exemplarily, the second reference transformation information may be obtained based on at least one transformation function (such as the S_{θ}(⋅) transformation function or the T_{θ}(⋅) transformation function involved in Formula 6).

Exemplarily, the implementation process of transforming the reference atomic feature hidden variable of the missing structure through the second reference transformation information to obtain the reference atomic feature information of the missing structure can be represented by Formula 6, where the x_{d+1:n} is used for representing the reference atomic feature information of the missing structure, the T_{θ}(z_{1:d}) and the S_{θ}(z_{1:d}) are used for representing the second reference transformation information obtained based on the z_{1:d}, the z_{1:d} is used for representing the reference atomic feature information of the molecule to be completed, and the z_{d+1:n} is used for representing the reference atomic feature hidden variable of the missing structure. The transformation does not change the dimension of the information, that is, the dimension of the reference atomic feature hidden variable of the missing structure is the same as the dimension of the reference atomic feature of the missing structure.

Exemplarily, both the reference atomic feature information of the molecule to be completed and the reference atomic feature information of the missing structure are matrices with the first reference dimension. The manner of obtaining the target atomic feature information based on the reference atomic feature information of the molecule to be completed and the reference atomic feature information of the missing structure may be as follows: elements at corresponding positions in the reference atomic feature information of the molecule to be completed and the reference atomic feature information of the missing structure are added, and the matrix obtained by adding is used as the target atomic feature information. Exemplarily, a Cartesian product between the reference atomic feature information of the molecule to be completed and the reference atomic feature information of the missing structure may also be used as the target atomic feature information.

In an exemplary aspect, the process of transforming the target atomic feature hidden variable needs to consider the constraints of the target chemical bond link information to ensure the reliability of the transformation process. In this case, constraint information of the target atomic feature hidden variable needs to be obtained based on the target chemical bond link information, and then, a target atomic completion model is called to transform the target atomic feature hidden variable under the constraint of the constraint information.

Exemplarily, the differential process of transforming the target atomic feature hidden variable under the constraint of the constraint information is reflected in the process of transforming the reference atomic feature hidden variable of the missing structure based on the reference atomic feature information of the molecule to be completed to obtain the reference atomic feature information of the missing structure, That is, the transformation of the reference atomic feature hidden variable of the missing structure is based on not only the reference atomic feature information of the molecule to be completed, but also the constraint information.

The manner of obtaining the constraint information of the target atomic feature hidden variable is not limited in the aspect of this disclosure, which may be set according to experiences or flexibly adjusted according to application scenarios. Exemplarily, the manner of obtaining the constraint information of the target atomic feature hidden variable may be as follows: the target chemical bond link information is used as the constraint information of the target atomic feature hidden variable. Exemplarily, the manner of obtaining the constraint information of the target atomic feature hidden variable may also be as follows: normalized processing is performed on the target chemical bond link information to obtain the constraint information of the target atomic feature hidden variable. The normalized processing of the target chemical bond link information is used for improving the normalization of the target chemical bond link information. The manner of the normalized processing may be set according to experiences or flexibly adjusted according to application scenarios, which is not limited in the aspect of this disclosure. For example, the normalized processing may be implemented by calling a graph normalization (Graphnorm for short) module.

Exemplarily, the molecule completion model includes an atomic completion model. Step 2033 can be implemented by calling the atomic completion model in the molecule completion model, that is, the atomic completion model in the molecule completion model is called to transform the target atomic feature hidden variable to obtain the target atomic feature information.

The atomic completion model can transform the atomic feature hidden variable of a molecule into the atomic feature information of the molecule, where the atomic feature hidden variable of the molecule is used for making assumptions about the features of atoms in the molecule, and the atomic feature information of the molecule is used for representing the features of atoms in the molecule. That is to say, the input of the atomic completion model is assumed information of the features of atoms in the molecule, and the output of the atomic completion model is representation information of the features of atoms in the molecule.

Exemplarily, the atomic completion model is an invertible model, that is, there is an inverse model of the atomic completion model. The inverse model of the atomic completion model can inversely transform the atomic feature information of a molecule into the atomic feature hidden variable of the molecule. That is to say, the input of the inverse model of the atomic completion model is representation information of the features of atoms in the molecule, and the output of the inverse model of the atomic completion model is assumed information of the features of atoms in the molecule.

The atomic completion model is obtained by training, and the model structure of the atomic completion model remains unchanged during the training process. The model structure of the atomic completion model can refer to the model structure of the atomic completion model in the aspect shown in FIG. 5, and will not be described herein again.

Step 2034: Determine, by the computer device, a completion result of the molecule to be completed based on the target chemical bond link information and the target atomic feature information.

The completion result of the molecule to be completed is a result capable of determining the molecule after completion of the molecule to be completed. The target chemical bond link information can indicate the chemical bond link situation between atoms in the molecule after completion of the molecule to be completed. The target atomic feature information can indicate the features of atoms in the molecule after completion of the molecule to be completed. A molecule after completion can be uniquely determined according to the chemical bond link situation between atoms in the molecule after completion and the features of atoms in the molecule after completion. Therefore, the completion result of the molecule to be completed can be obtained based on the target chemical bond link information and the target atomic feature information.

Exemplarily, the manner of obtaining the completion result of the molecule to be completed based on the target chemical bond link information and the target atomic feature information may be as follows: information including the target chemical bond link information and the target atomic feature information is used as the completion result of the molecule to be completed.

Exemplarily, the manner of obtaining the completion result of the molecule to be completed based on the target chemical bond link information and the target atomic feature information may also be as follows: a molecular formula or a graph structure of the molecule to be completed is determined based on the target chemical bond link information and the target atomic feature information, and the molecular formula or the graph structure is used as the completion result of the molecule to be completed.

Exemplarily, the process of obtaining the completion result of the molecule to be completed based on step 2031 to step 2034 can be represented as:

input: Z_{A}_{^{S}}=(A^{S}, Z_{A}_{^{L}}), Z_{B}_{^{S}}=(B^{S}, Z_{B}_{^{L}}), an inverse model f_{A}_{^{R}}_{|B}_{^{R}} of the target atomic completion model, and an inverse model f_{B}_{^{R}} of the target chemical bond completion model, where the A^{S} and the B^{S} represent the atomic feature information and the chemical bond link information of the molecule G^{s} to be completed, and the Z_{A}_{^{L}} and the Z_{B}_{^{L}} represent the atomic feature hidden variable and the chemical bond link hidden variable of the missing structure in the molecule to be completed; the Z_{A}_{^{S}} represents the target atomic feature hidden variable of the molecule after completion; the Z_{B}_{^{S}} represents the target chemical bond link hidden variable of the molecule after completion;
1. (Z_{A}_{^{S}}, Z_{B}_{^{S}})=Z_{G}_{^{S}}//obtain information Z_{G}_{^{S}} to be processed, the information Z_{G}_{^{S}} to be processed including the target atomic feature hidden variable Z_{A}_{^{S}} and the target chemical bond link hidden variable Z_{B}_{^{S}} of the molecule after completion
2. B^{R}=f_{B}_{^{R}}⁻¹(Z_{B}_{^{S}})//call a target chemical bond completion model f_{B}_{^{R}}⁻¹ to transform the target chemical bond link hidden variable Z_{B}_{^{S}} to obtain target chemical bond link information B^{R}
3. \hat{B}^{R}=Graphnorm(B^{R})//perform normalized processing on the target chemical bond link information B^{R} based on the Graphnorm module to obtain constraint information B^{R}
4. A^{R}=f_{A}_{^{R}}_{|B}_{^{R}}⁻¹(Z_{A}_{^{S}}|\hat{B}^{R})//call a target atomic completion model f_{A}_{^{R}}_{|B}_{^{R}}⁻¹ to transform the target atomic feature hidden variable Z_{A}_{^{S}} under the constraint of the constraint information \hat{B}^{R} to obtain target atomic feature information A^{R}
output: a completion result G^{R}=(A^{R}, B^{R}).

Exemplarily, the input of the flow-based generative model is a molecule GS=(V^{S}, B^{S}, A^{S}) to be completed, and the output of the flow-based generative model is a molecule G^{R}=(V^{R}, B^{R}, A^{R}) after completion (that is, a reactant molecule). V^{S}, B^{S} and A^{S} respectively represent an atom set, chemical bond link information and atomic feature information of the molecule to be completed, and V^{R}, B^{R} and A^{R} respectively represent an atom set, chemical bond link information and atomic feature information of the reactant molecule. Exemplarily, the flow-based generative model is a non-autoregressive generative model capable of generating a completion result once. Compared with an autoregressive generative model, the flow-based generative model has a higher generation speed and is configured to improve the efficiency of predicting the reactant molecule.

Step 2031 to step 2034 only take the molecule completion model being a flow-based generative model as an example to introduce the implementation process of calling the molecule completion model to complete the molecule to be completed, which is not limited in the aspect of this disclosure. In some aspects, the molecule completion model may also be other types of generative models, such as a variational autoencoder (VAE) generative model or a generative adversarial model (GAN), which is not limited in the aspect of this disclosure. Certainly, in some aspects, the molecule completion model may also be a convolutional neural network (CNN) model, or the like. In different cases of molecule completion models, the processes of calling the molecule completion model to complete the molecule to be completed to obtain the completion result of the molecule to be completed are also different, which will not be introduced one by one in the aspect of this disclosure.

Exemplarily, the process of calling the molecule completion model to complete the molecule to be completed to obtain the completion result of the molecule to be completed may be as follows: the molecule completion model is called to extract the features of the molecule to be completed; the features of the molecule after completion of the molecule to be completed are predicted based on the features of the molecule to be completed; and the completion result of the molecule to be completed is obtained based on the features of the molecule after completion of the molecule to be completed.

Exemplarily, the process of completing at least one molecule to be completed can be regarded as a second stage in the reactant molecule prediction process. In the second stage, atoms and chemical bonds are added on the basis of the molecule to be completed obtained in the first stage, and the molecule to be completed is completed (or reduced) into the original reactant molecule. The operation in this stage can be considered as modeling probability distribution P(G^{R}|{Gₕ^{S}}ₕ₌₁^{H}), which can be solved as a conditional generation problem, where the G^{R} represents a reactant molecule. As mentioned above, the aspect of this disclosure assumes that one molecule to be completed corresponds to one reactant molecule, the probability distribution that needs to be established can be represented as P(G_{c}^{R}|Gₕ^{S}), where the G_{c}^{R} represents the reactant molecule of the Gₕ^{S}, and the Gₕ^{S} represents the h-th (h is an integer not less than 1) molecule to be completed. Exemplarily, the second stage is shown in FIG. 4 (2). The molecule completion model is called to respectively complete two molecules to be completed to obtain two reactant molecules of the product molecule.

According to the method for predicting reactant molecules provided in the aspect of this disclosure, the prediction process of the reactant molecule is implemented by the molecule completion model, and the molecule completion model is obtained by training based on the sample compound molecule and the sample molecule to be completed. The sample molecule to be completed is obtained by masking the sub-structure in the sample compound molecule, that is, the data for the training process of the molecule completion model is the data obtained on the basis of the sample compound molecule, this training process is a self-supervised training process based on the sample compound molecule, and this self-supervised training process does not require attention to whether the sample compound molecule is a compound in a known synthesis reaction, so that this self-supervised training process is not limited by the known synthesis reaction, and the molecule completion model trained by the training process has a stronger generalization ability, which is beneficial for expanding adaptation scenarios so as to improve the prediction reliability and prediction accuracy of the reactant molecule.

An aspect of this disclosure provides a method for training molecule completion models. The method can be applied to the foregoing implementation environment shown in FIG. 1. The method for training molecule completion models is performed by a computer device. The computer device may be the terminal 11 or the server 12, which is not limited in the aspect of this disclosure. As shown in FIG. 5, a method for training molecule completion models provided in an aspect of this disclosure includes step 501 and step 502.

In step 501, the computer device obtains a sample compound molecule and a sample molecule to be completed, the sample molecule to be completed being obtained by masking a sub-structure in the sample compound molecule.

The sample compound molecule is a compound molecule for training the molecule completion model once. There may be one or more sample compound molecules, which is not limited in the aspect of this disclosure. Exemplarily, the sample compound molecule may be extracted from any data set that includes compound molecules. Exemplarily, a data set that includes compound molecules may be a data set that includes known synthesis reactions or a data set that does not include known synthesis reactions, which is not limited in the aspect of this disclosure. That is to say, the sample compound molecule has higher obtaining flexibility and is not limited to the data set that includes known synthesis reactions, thus being beneficial for improving the generalization ability of a model obtained by training. Exemplarily, because the training process of a model does not rely on a label of a sample compound, a data set that includes compounds may be an unlabeled data set.

After the sample compound molecule is obtained, the sample molecule to be completed of the sample compound molecule can be obtained, where the sample molecule to be completed is obtained by masking the sub-structure in the sample compound molecule. Exemplarily, masking the sub-structure in the sample compound molecule refers to hiding the sub-structure in the sample compound molecule. After the sub-structure in the sample compound molecule is masked, the original state of the sub-structure in the sample compound molecule cannot be obtained. The manner of masking the sub-structure in the sample compound molecule may be set according to experiences or flexibly adjusted according to actual application scenarios, as long as the sub-structure in the sample compound molecule can be hidden. Exemplarily, the manner of masking the sub-structure in the sample compound molecule may be implemented by replacing the sub-structure in the sample compound molecule with a specific structure, or by covering the sub-structure in the sample compound molecule. Exemplarily, the specific structure refers to a structure that is different from a real structure of a compound molecule, so as to distinguish a masked part from an unmasked part.

The sub-structure in the sample compound molecule refers to a part of the sample compound molecule. The aspect of this disclosure does not limit the complexity of the masked sub-structure in the sample compound molecule. Exemplarily, the masked sub-structure in the sample compound molecule may be one atom in the sample compound molecule, one chemical bond in the sample compound molecule, one structure composed of at least one atom and at least one chemical bond in the sample compound molecule, or the like.

Exemplarily, the case where the masked sub-structure in the sample compound molecule is one atom in the sample compound molecule may be shown in FIG. 6 (1), the case where the masked sub-structure in the sample compound molecule is one chemical bond in the sample compound molecule may be shown in FIG. 6 (2), and the case where the masked sub-structure in the sample compound molecule is one structure composed of at least one atom and at least one chemical bond in the sample compound molecule may be shown in FIG. 6 (3). In FIG. 6, parts marked with question marks and shielded parts are masked sub-structures.

In an exemplary aspect, in a case that the masked sub-structure in the sample compound molecule is one atom in the sample compound molecule or one chemical bond in the sample compound molecule, the process of training the molecule completion model can be regarded as a process of training the molecule completion model based on a reconstruction task of single atoms or single chemical bonds. The reconstruction task based on single atoms or single chemical bonds is a relatively simple task, which is beneficial for improving the convergence speed of model training. Exemplarily, training the molecule completion model based on the reconstruction task of single atoms or single chemical bonds can be regarded as a multi-classification problem, and the multi-classification problem is used for predicting the types of masked single atoms or single chemical bonds.

In an exemplary aspect, a structure composed of at least one atom and at least one chemical bond can be referred to as a sub-graph. In a case that the masked sub-structure in the sample compound molecule is a structure composed of at least one atom and at least one chemical bond in the sample compound molecule, the level of masking the sample compound can be referred to as a sub-graph level, and this level of masking is beneficial for improving the completion ability of the model.

Exemplarily, the masked sub-structure in the sample compound molecule may be selected from the sample compound molecule according to experiences. For example, any central atom may be selected from the sample compound molecule, g (g is an integer not less than 0) jumps are performed from the central atom, and the sub-structure covered by the g jumps is used as the masked sub-structure in the sample compound molecule. This manner of selecting the masked sub-structure in the sample compound molecule is relatively simple.

Exemplarily, the masked sub-structure in the sample compound molecule may also be selected from the sample compound molecule by referring to a candidate structure set. In this case, the masked sub-structure in the sample compound molecule is a structure belonging to the candidate structure set in the sample compound. The structure belonging to the candidate structure set refers to a structure that constitutes the candidate structure set. The candidate structure set is a set of structures with confidences meeting selection conditions. A structure with a confidence meeting selection conditions refers to a structure with a higher confidence. Selecting the masked sub-structure from the sample compound molecule by referring to the structure with a higher confidence is beneficial for improving the rationality of the masked sub-structure and avoiding the collapse of the overall structure, thus improving the completion performance of the model.

Confidences meeting selection conditions may be set according to experiences or flexibly adjusted according to application scenarios, which are not limited in the aspect of this disclosure. In an exemplary aspect, structures with confidences meeting selection conditions may include difference structures between product molecules and reactant molecules in known synthesis reactions. A known synthesis reaction may be extracted from an inverse synthesis data set, and the inverse synthesis data set may be selected according to experiences. For example, an inverse synthesis data set is a USPTO-50K data set which includes fifty thousand inverse synthesis reactions, and each inverse synthesis reaction is a known synthesis reaction.

In an exemplary aspect, structures with confidences meeting selection conditions may further include motifs (which can be referred to as sequence motifs and are basic structures for constituting any type of feature sequence), functional groups with an occurrence frequency greater than a frequency threshold, structures obtained by splitting reference molecules according to BRICS (an algorithm for splitting molecules), and the like. Exemplarily, the occurrence frequency may be an occurrence frequency in some articles or journals, an occurrence frequency in inverse synthesis data sets, or the like. Reference molecules may be selected according to experiences.

Exemplarily, the candidate structure set may also be referred to as a sub-structure dictionary. A construction manner of the sub-structure dictionary may be flexibly selected, as long as an internal structure is a structure with a confidence meeting selection conditions. In different construction manners, the average size and average occurrence frequency of structures in the sub-structure dictionary may be different.

Exemplarily, in a case that the masked sub-structure in the sample compound molecule is a structure belonging to the candidate structure set in the sample compound, the implementation process of obtaining the sample molecule to be completed may be as follows: any chemical bond is selected from the sample compound molecule, the chemical bond is cut to obtain two structures, and the structure with a smaller number of atoms in the two structures is matched with the candidate structure set; if the matching is successful (that is, the structure with a smaller number of atoms belongs to the candidate structure set), it is determined that a cutting solution of the structure with a smaller number of atoms is reasonable; and the structure with a smaller number of atoms is used as the masked sub-structure in the sample compound molecule, and the masked sub-structure is masked to obtain the sample molecule to be completed. Exemplarily, if the matching fails, (that is, the structure with a smaller number of atoms does not belong to the candidate structure set), a chemical bond is selected again and cut. This implementation process can be referred to as a process of obtaining the sample molecule to be completed based on molecular decomposition. This molecular decomposition can ensure that the masked sub-structure and the remaining parts retain meaningful structures, thus reducing the completion (or called reconstruction) difficulty.

Exemplarily, different chemical bonds in the same sample compound molecule are selected and cut, and the obtained structures with a smaller number of atoms are different. For example, as shown in FIG. 7, if a chemical bond 1 in the sample compound molecule is selected and cut, the obtained structure with a smaller number of atoms is shown in 701; and if a chemical bond 2 in the sample compound molecule is selected and cut, the obtained structure with a smaller number of atoms is shown in 702.

For the same sample compound molecule, different molecules to be completed can be obtained under different mask solutions. The sample compound molecule and each molecule to be completed can constitute a data pair. The model training process in the aspect of this disclosure is performed on the basis of the data pair. That is to say, the sample molecule to be completed in the aspect of this disclosure refers to any molecule to be completed of the sample compound molecule.

Step 502: Determine, by the computer device, training loss based on the sample compound molecule, the sample molecule to be completed, and a molecule completion model; and update model parameters of the molecule completion model based on the training loss to obtain a molecule completion model after training. For example, the model parameters of the molecule completion model are updated based on the training loss to obtain a trained molecule completion model.

The training loss is used for providing supervision information for the updating of the model parameters of the molecule completion model. The implementation manner of obtaining the training loss based on the sample compound molecule, the sample molecule to be completed and the molecule completion model is related to the type of the molecule completion model, which is not limited in the aspect of this disclosure.

In a possible implementation, the implementation manner of determining the training loss by the computer device based on the sample compound molecule, the sample molecule to be completed and the molecule completion model includes step 5021 to step 5025.

Step 5021: Obtain, by the computer device, sample atomic feature information and sample chemical bond link information of the sample compound molecule.

The sample atomic feature information of the sample compound molecule is used for representing the features of atoms in the sample compound molecule, and the sample chemical bond link information of the sample compound molecule is used for representing the chemical bond link situation between atoms in the sample compound molecule. The principle of obtaining the sample atomic feature information and the sample chemical bond link information of the sample compound molecule is the same as the principle of obtaining the atomic feature information and the chemical bond link information of the product molecule in the aspect shown in FIG. 2, and will not be described herein again. In an exemplary aspect, the sample chemical bond link information of the sample compound molecule is a matrix with the first reference dimension, and the sample atomic feature information of the sample compound molecule is a matrix with the second reference dimension.

Step 5022: Determine, by the computer device, atomic mask information and chemical bond mask information based on a difference between the sample compound molecule and the sample molecule to be completed.

The atomic mask information indicates the masking situation of atoms in the sample compound molecule, for example, which atoms are masked, and which atoms are not masked. The chemical bond mask information indicates the masking situation of chemical bonds between atoms in the sample compound molecule, for example, which chemical bonds are masked, and which chemical bonds are not masked. Because the sample molecule to be completed is obtained by masking the sub-structure in the sample compound, the atomic mask information and the chemical bond mask information can be determined by comparing the difference structures between the sample compound molecule and the sample molecule to be completed.

Exemplarily, the dimension of the atomic mask information is the same as the dimension of the sample atomic feature information, for example, both the atomic mask information and the sample atomic feature information are matrices with the second reference dimension. The value of an element at any position in the atomic mask information indicates the masking situation of an element at the same position in the sample atomic feature information. For example, in a case that the value of an element at any position in the atomic mask information is 0, this indicates that an element at the same position in the sample atomic feature information is masked; and in a case that the value of an element at any position in the atomic mask information is 1, this indicates that an element at the same position in the sample atomic feature information is not masked.

Exemplarily, the dimension of the chemical bond mask information is the same as the dimension of the sample chemical bond link information, for example, both the chemical bond mask information and the sample chemical bond link information are matrices with the first reference dimension. The value of an element at any position in the chemical bond mask information indicates the masking situation of an element at the same position in the sample chemical bond link information. For example, in a case that the value of an element at any position in the chemical bond mask information is 0, this indicates that an element at the same position in the sample chemical bond link information is masked; and in a case that the value of an element at any position in the chemical bond mask information is 1, this indicates that an element at the same position in the sample chemical bond link information is not masked.

Step 5023: Calls, by the computer device, the molecule completion model to perform an inverse transformation on the sample chemical bond link information based on the chemical bond mask information to obtain a sample chemical bond link hidden variable.

The sample chemical bond link hidden variable is used for making assumptions about the chemical bond link situation between atoms in the sample compound molecule. The molecule completion model is an invertible model which can not only transform the chemical bond link hidden variable of a molecule into the chemical bond link information of the molecule, but also inversely transform the chemical bond link information of the molecule into the chemical bond link hidden variable of the molecule. Because during the training process of a model, the sample chemical bond link information of the sample compound molecule is known relatively accurate information, the molecule completion model is called to implement an inverse transformation of the sample chemical bond link information.

In a possible implementation, the process of calling the molecule completion model by the computer device to perform an inverse transformation on the sample chemical bond link information based on the chemical bond mask information to obtain the sample chemical bond link hidden variable includes step 50231 to step 50233.

Step 50231: Call, by the computer device, the molecule completion model to determine first chemical bond link information and second chemical bond link information based on the chemical bond mask information and the sample chemical bond link information, the first chemical bond link information being chemical bond link information of the sample molecule to be completed, and the second chemical bond link information being chemical bond link information of the sub-structure.

The first chemical bond link information represents the chemical bond link situation between atoms in the sample molecule to be completed, and the second chemical bond link information represents the chemical bond link situation between atoms in the masked sub-structure. The chemical bond mask information can indicate the chemical bond masking situation between atoms in the sample compound molecule, masked chemical bonds are chemical bonds between atoms in the masked sub-structure, and unmasked chemical bonds are chemical bonds between atoms in the sample molecule to be completed, so the first chemical bond link information of the sample molecule to be completed and the second chemical bond link information of the sub-structure can be obtained based on the chemical bond mask information and the sample chemical bond link information of the sample compound molecule.

Exemplarily, the manner of determining the first chemical bond link information and the second chemical bond link information based on the chemical bond mask information and the sample chemical bond link information may be as follows: the information related to the unmasked chemical bonds indicated by the chemical bond mask information in the sample chemical bond link information is retained, and other information is set to 0 to obtain the first chemical bond link information; and the information related to the masked chemical bonds indicated by the chemical bond mask information in the sample chemical bond link information is retained, and other information is set to 0 to obtain the second chemical bond link information. In this manner, both the dimension of the first chemical bond link information and the dimension of the second chemical bond link information are the same as the dimension of the matrix of the sample chemical bond link information, for example, both the first chemical bond link information and the second chemical bond link information are matrices with the first reference dimension.

Step 50232: Perform, by the computer device, an inverse transformation on the second chemical bond link information based on the first chemical bond link information to obtain a chemical bond link hidden variable of the sub-structure.

The chemical bond link hidden variable of the sub-structure is used for making assumptions about the chemical bond link situation between atoms in the sub-structure. Exemplarily, the chemical bond link hidden variable of the sub-structure is a variable following the known probability distribution. The known probability distribution may be set according to experiences or flexibly adjusted according to application scenarios. For example, the known probability distribution is Gaussian distribution or uniform distribution.

In an exemplary aspect, the implementation process of performing an inverse transformation on the second chemical bond link information based on the first chemical bond link information to obtain the chemical bond link hidden variable of the sub-structure includes the steps of: obtaining first sample transformation information based on the first chemical bond link information; and performing an inverse transformation on the second chemical bond link information through the first sample transformation information to obtain the chemical bond link hidden variable of the sub-structure. Exemplarily, the first sample transformation information may be obtained based on at least one transformation function (such as a S_{θ}(⋅) transformation function or a T_{θ}(⋅) transformation function). The inverse transformation does not change the dimension of the information, that is, the dimension of the chemical bond link hidden variable of the sub-structure is the same as the dimension of the second chemical bond link information.

Step 50233: Determine, by the computer device, the sample chemical bond link hidden variable based on the first chemical bond link information and the chemical bond link hidden variable of the sub-structure.

Exemplarily, both the first chemical bond link information and the chemical bond link hidden variable of the sub-structure are matrices with the first reference dimension. The manner of obtaining the sample chemical bond link hidden variable based on the first chemical bond link information and the chemical bond link hidden variable of the sub-structure may be as follows: elements at corresponding positions in the first chemical bond link information and the chemical bond link hidden variable of the sub-structure are added, and the matrix obtained by adding is used as the sample chemical bond link hidden variable. Exemplarily, a Cartesian product between the first chemical bond link information and the chemical bond link hidden variable of the sub-structure may also be used as the sample chemical bond link hidden variable.

Exemplarily, the process of obtaining the sample chemical bond link hidden variable may be implemented based on Formula 7 and Formula 8:

$${Z}_{{B}_{1}^{R}} = {B}_{1}^{R} \qquad ( Formula 7 )$$

$${Z}_{{B}_{2}^{R}} = {B}_{2}^{R} ⊙ Sigmoid ( {S}_{θ} ( {B}_{1}^{R} ) ) + {T}_{θ} ( {B}_{1}^{R} ) \qquad ( Formula 8 )$$

where the B₁^{R} and the Z_{B}_{₁}_{^{R}} represent the first chemical bond link information;
the B₂^{R} represents the second chemical bond link information; the S_{θ}(B₁^{R}) and the T_{θ}(B₁^{R}) represent the first sample transformation information; the BROSigmoid(S_{θ}(B^{R}))+T_{θ}(B^{R}) represents an operation mode of performing an inverse transformation on the second chemical bond link information based on the first sample transformation information; and the Z_{B}_{₂}_{^{R}} represents the chemical bond link hidden variable of the sub-structure. The Z_{B}_{₁}_{^{R}} and the Z_{B}_{₂}_{^{R}} are two components of the sample chemical bond link hidden variable. Exemplarily, Formula 7 is used for keeping the first chemical bond link information B^{R} of the sample molecule to be completed unchanged, and Formula 8 is used for transforming the second chemical bond link information B₂^{R} of the masked sub-structure into a hidden variable following Gaussian distribution. The S_{θ} and the T_{θ} can adopt neural network structures, such as graph neural network structures.

Exemplarily, the molecule completion model includes a chemical bond completion model. The chemical bond completion model is a flow-based generative model, that is, the chemical bond completion model is configured to complete the chemical bond link information in a flow-based generative manner. Exemplarily, the molecule completion model may also be referred to as a synthon flow model, and the chemical bond completion model may also be referred to as a synthon bond flow (SB Flow for short) model.

The chemical bond completion model is an invertible model, and the relationship between the inverse model of the chemical bond completion model and the chemical bond completion model is as follows: the input of the inverse model of the chemical bond completion model is the output of the chemical bond completion model, and the output of the inverse model of the chemical bond completion model is the input of the chemical bond completion model. Exemplarily, the dimensions of the information of the input and the output of the chemical bond completion model are the same. Exemplarily, the input of the chemical bond completion model is assumed information of the chemical bond link situation between atoms in the molecule, and the output of the chemical bond completion model is representation information of the chemical bond link situation between atoms in the molecule. That is to say, the input of the inverse model of the chemical bond model is representation information of the chemical bond link situation between atoms in the molecule, and the output of the inverse model of the chemical bond model is assumed information of the chemical bond link situation between atoms in the molecule.

Because the current input information is the sample chemical bond link information (that is, the representation information of the chemical bond link situation between atoms in the sample compound molecule), step 5023 can be implemented by calling the inverse model of the chemical bond completion model in the molecule completion model. That is to say, the inverse model of the chemical bond completion model in the molecule completion model is called to perform an inverse transformation on the sample chemical bond link information based on the chemical bond mask information to obtain the sample chemical bond link hidden variable.

Exemplarily, the chemical bond completion model includes at least one chemical bond completion module, and each chemical bond completion module has the same structure. The aspect of this disclosure takes the chemical bond completion model including one chemical bond completion module as an example for description.

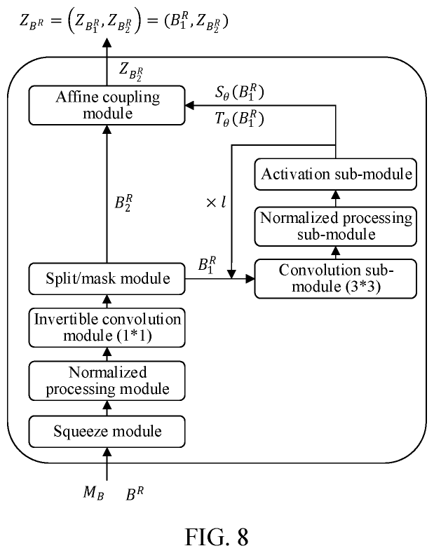

Exemplarily, the structure of the chemical bond completion model may be shown in FIG. 8. The chemical bond completion model includes a squeeze module, a normalized processing (Actnorm) module, an invertible convolution module, a split/mask module, an affine coupling module, and at least one transformation information obtaining module. Exemplarily, the transformation information obtaining module includes a convolution sub-module, a normalized processing (Batchnorm) sub-module, and an activation (Relu) sub-module. In FIG. 8, the number of transformation information obtaining modules is 1 (1 is an integer not less than 1), the convolution kernel of the invertible convolution module is 1*1, and the convolution kernel of the convolution sub-module in the transformation information obtaining module is 3*3. Exemplarily, the squeeze module is configured to transform the dimension of the chemical bond link information of the input; the normalized processing module is configured to perform normalized processing on the information; the invertible convolution module is configured to rearrange type dimensions in the chemical bond link information; the split/mask module is configured to split the sample chemical bond link information of the sample compound into two parts (the first chemical bond link information and the second chemical bond link information) based on the chemical bond mask information; the affine coupling module is configured to implement an inverse transformation of the second chemical bond link information; and the/transformation information obtaining modules are configured to obtain the first sample transformation information.

Exemplarily, under the structure of the chemical bond completion model shown in FIG. 8, the process of obtaining the sample chemical bond link hidden variable may be as follows: chemical bond mask information M_{B} and sample chemical bond link information B^{R} are inputted into the chemical bond completion model, and are sequentially processed by the squeeze module, the normalized processing module, the invertible convolution module and the split/mask module to obtain first chemical bond link information B₁^{R} and second chemical bond link information B₂^{R}. The l transformation information obtaining modules (each transformation information obtaining module includes a convolution sub-module, a normalized processing sub-module, and an activation sub-module) are configured to process the first chemical bond link information B^{R} to obtain first sample transformation information S_{θ}(B₁^{R}) and T_{θ}(B₁^{R}), and then, the affine coupling module performs an inverse transformation on the second chemical bond link information B₂^{R} through the first sample transformation information S_{θ}(B^{R}) and T_{θ}(B₁^{R}) to obtain a chemical bond link hidden variable Z_{B}_{₂}_{^{R}} of the sub-structure; and a sample chemical bond link hidden variable Z_{B}_{^{R}} is obtained based on the first chemical bond link information B₁^{R} and the chemical bond link hidden variable Z_{B}_{₂}_{^{R}} of the sub-structure.

The structure of the chemical bond completion model shown in FIG. 8 is only an example, which is not limited in the aspect of this disclosure. That is to say, the structure of the chemical bond completion model may further include more or less modules.

In an exemplary aspect, the process of performing an inverse transformation on the sample chemical bond link information based on the chemical bond mask information may be a process of performing an inverse transformation on the sample chemical bond link information based on the chemical bond mask information directly, or a process of performing an inverse transformation on the processed chemical bond link information based on the chemical bond mask information, where the processed chemical bond link information may be obtained by calling a GLOW module (an information processing module) to process the sample chemical bond link information.

Step 5024: Call, by the computer device, the molecule completion model to perform an inverse transformation on the sample atomic feature information based on the atomic mask information to obtain a sample atomic feature hidden variable.

The sample atomic feature hidden variable is used for making assumptions about the features of atoms in the sample compound molecule. The molecule completion model is an invertible model which can not only transform the atomic feature hidden variable of a molecule into the atomic feature information of the molecule, but also inversely transform the atomic feature information of the molecule into the atomic feature hidden variable of the molecule. Because during the training process of a model, the sample atomic feature information of the sample compound molecule is known relatively accurate information, the molecule completion model is called to implement an inverse transformation of the sample atomic feature information.

In a possible implementation, the process of calling the molecule completion model by the computer device to perform an inverse transformation on the sample atomic feature hidden variable based on the atomic mask information to obtain the sample atomic feature hidden variable includes step 50241 to step 50243.

Step 50241: Call, by the computer device, the molecule completion model to determine first atomic feature information and second atomic feature information based on the atomic mask information and the sample atomic feature information, the first atomic feature information being atomic feature information of the sample molecule to be completed, and the second atomic feature information being atomic feature information of the sub-structure.

The first atomic feature information is used for representing the features of atoms in the sample molecule to be completed, and the second atomic feature information is used for representing the features of atoms in the masked sub-structure. The atomic mask information can indicate the masking situation of atoms in the sample compound molecule, masked atoms are atoms in the masked sub-structure, and unmasked atoms are atoms in the sample molecule to be completed, so the first atomic feature information of the sample molecule to be completed and the second atomic feature information of the sub-structure can be obtained based on the atomic mask information and the sample atom feature of the sample compound molecule.

Exemplarily, the manner of obtaining the first atomic feature information of the sample molecule to be completed and the second atomic feature information of the sub-structure based on the atomic mask information and the sample atomic feature information may be as follows: the information related to the unmasked atoms indicated by the atomic mask information in the sample atomic feature information is retained, and other information is set to 0 to obtain the first atomic feature information; and the information related to the masked atoms indicated by the atomic mask information in the sample atomic feature information is retained, and other information is set to 0 to obtain the second atomic feature information. In this manner, both the dimension of the first atomic feature information and the dimension of the second atomic feature information are the same as the dimension of the matrix of the sample atomic feature information, for example, both the first atomic feature information and the second atomic feature information are matrices with the second reference dimension.

Step 50242: Perform, by the computer device, an inverse transformation on the second atomic feature information based on the first atomic feature information to obtain an atomic feature hidden variable of the sub-structure.

The atomic feature hidden variable of the sub-structure is used for making assumptions about the features of atoms in the sub-structure. Exemplarily, the atomic feature hidden variable of the sub-structure is a variable following the known probability distribution. The known probability distribution may be set according to experiences or flexibly adjusted according to application scenarios. For example, the known probability distribution is Gaussian distribution or uniform distribution.

In an exemplary aspect, the implementation process of performing an inverse transformation on the second atomic feature information based on the first atomic feature information to obtain the atomic feature hidden variable of the sub-structure includes the steps of: obtaining second sample transformation information based on the first atomic feature information; and performing an inverse transformation on the second atomic feature information through the second sample transformation information to obtain the atomic feature hidden variable of the sub-structure. Exemplarily, the second sample transformation information may be obtained based on at least one transformation function (such as a S_{θ}(⋅) transformation function or a T_{θ}(⋅) transformation function). The inverse transformation does not change the dimension of the information, that is, the dimension of the atomic feature hidden variable of the sub-structure is the same as the dimension of the second atomic feature information.

In an exemplary aspect, the process of performing an inverse transformation on the second atomic feature information based on the first atomic feature information needs to consider the constraints of the sample chemical bond link information to ensure the reliability of the inverse transformation process. In this case, sample constraint information needs to be obtained based on the sample chemical bond link information, and then, an inverse transformation is performed on the second atomic feature information based on the first atomic feature information and the sample constraint information to obtain the atomic feature hidden variable of the sub-structure. In this case, the second sample transformation information is obtained by comprehensively considering the first atomic feature information and the sample constraint information.

The manner of obtaining the sample constraint information is not limited in the aspect of this disclosure, which may be set according to experiences or flexibly adjusted according to application scenarios. Exemplarily, the manner of obtaining the sample constraint information may be as follows: the sample chemical bond link information is used as the sample constraint information. Exemplarily, the manner of obtaining the sample constraint information may also be as follows: normalized processing is performed on the sample chemical bond link information to obtain the sample constraint information. The normalized processing of the sample chemical bond link information is used for improving the normalization of the sample chemical bond link information. The manner of the normalized processing may be set according to experiences or flexibly adjusted according to application scenarios, which is not limited in the aspect of this disclosure. For example, the normalized processing may be implemented by calling a graph normalization module.

Step 50243: Obtain, by the computer device, a sample atomic feature hidden variable based on the first atomic feature information and the atomic feature hidden variable of the sub-structure.

Exemplarily, both the first atomic feature information and the atomic feature hidden variable of the sub-structure are matrices with the second reference dimension. The manner of obtaining the sample atomic feature hidden variable based on the first atomic feature information and the atomic feature hidden variable of the sub-structure may be as follows: elements at corresponding positions in the first atomic feature information and the atomic feature hidden variable of the sub-structure are added, and the matrix obtained by adding is used as the sample atomic feature hidden variable. Exemplarily, a Cartesian product between the first atomic feature information and the atomic feature hidden variable of the sub-structure may also be used as the sample atomic feature hidden variable.

Exemplarily, the process of obtaining the sample atomic feature hidden variable may be implemented based on Formula 9 and Formula 10:

$${Z}_{{A}_{1}^{R} \vert {\hat{B}}^{R}} = {A}_{1}^{R} \qquad ( Formula 9 )$$

$${Z}_{{A}_{2}^{R} \vert {\hat{B}}^{R}} = {A}_{2}^{R} ⊙ Sigmoid ( {S}_{θ} ( {A}_{1}^{R} \vert {\hat{B}}^{R} ) ) + {T}_{θ} ( {A}_{1}^{R} \vert {\hat{B}}^{R} ) \qquad ( Formula 10 )$$

where the A₁^{R} and the Z_{A}_{₁}_{^{R}}_{|\hat{B}}_{^{R}} represent the first atomic feature information; the \hat{B}^{R} represents the sample constraint information; the A₂^{R} represents the second atomic feature information; the S_{θ}(A₁^{R}|\hat{B}^{R}) and the T_{θ}(A₁^{R}|\hat{B}^{R}) represent the second sample transformation information; the A₂^{R}⊙Sigmoid (S_{θ}(A₁^{R}|\hat{B}^{R}))+T_{θ}(A₁^{R}|\hat{B}^{R}) represents an operation mode of performing an inverse transformation on the second atomic feature information based on the second sample transformation information; and the Z_{A}_{₂}_{^{R}}_{|\hat{B}}_{^{R}} represents the atomic feature hidden variable of the sub-structure. The Z_{A}_{₁}_{^{R}}_{|\hat{B}}_{^{R}} and the Z_{A}_{₂}_{^{R}}_{|\hat{B}}_{^{R}} are two components of the sample atomic feature hidden variable.

Exemplarily, Formula 9 is used for keeping the first atomic feature information A₁^{R} of the sample molecule to be completed unchanged, Formula 10 is used for transforming the second atomic feature information A₂^{R} of the masked sub-structure into a hidden variable following Gaussian distribution, and Formula 10 takes the first atomic feature information A₁^{R} and the sample constraint information \hat{B}^{R} obtained based on the sample chemical bond link information B^{R} as input conditions. The S_{θ} and the T_{θ} can adopt neural network structures, such as graph neural network structures. The output dimensions of the Sθ and the T_{θ} are both the same as the dimension of the second atomic feature information A₂^{R}. The processing logic of the S_{θ} and the T_{θ} may be represented by Formula 11:

$${h}_{A} = Graphconv ( {A}_{1}^{R} ) = {\sum }_{i = 1}^{C} {\hat{B}}_{i}^{R} ( {M}_{A} ⊙ {A}^{R} ) {W}_{i} + ( {M}_{A} ⊙ {A}^{R} ) {W}_{0} \qquad ( Formula 11 )$$

where the h_{A} represents an output structure of a S_{θ} or T_{θ} function (that is, S_{θ}(A₁^{R}|\hat{B}^{R}) or T_{θ}(A₁^{R}|\hat{B}^{R})); and the M_{A}∈{0,1} represents the atomic mask information for masking the atomic feature information of a non-sample molecule to be completed (that is, the masked sub-structure) into 0 and keeping the atomic feature information of the sample molecule to be completed unchanged. The dimension of the M_{A} is the same as the dimension of the sample atomic feature information, and the M_{A} includes a mask value of each element in the sample atomic feature information. Exemplarily, for all j (j is any dimension in all dimensions of the sub-feature information of any atom), if vₐ∈V^{S}, M_{A}[a, j]=1, otherwise, M_{A}[a, j]=0, where the vₐ∈V^{S} represents that an atom a is an atom in an atom set V^{S} composed of atoms in the sample molecule to be completed, and the M[a, j] represents a mask value of an element with the j-th dimension in the sub-feature information of the atom a.

The A₁^{R} represents the first atomic feature information, and the A₁^{R} can be obtained by the M_{A}⊙A^{R} operation on the basis of the M_{A}. The \hat{B}ᵢ^{R} represents the information related to a chemical bond type i in the sample constraint information obtained based on the sample chemical bond link information, i is an integer not less than 1 and not greater than C, and C (C is an integer not less than 1) represents the number of candidate chemical bond types. Graphconv( ) represents a graph neural network structure; and the Wᵢ and the W₀ represent parameters of the graph neural network structure.

Exemplarily, the molecule completion model includes an atomic completion model. The atomic completion model is a flow-based generative model, that is, the atomic completion model is configured to complete the atomic feature information in a flow-based generative manner. Exemplarily, the atomic completion model may also be referred to as a synthon graph flow (SG Flow for short) model.

The atomic completion model is an invertible model, and the relationship between the inverse model of the atomic completion model and the atomic completion model is as follows: the input of the inverse model of the atomic completion model is the output of the atomic completion model, and the output of the inverse model of the atomic completion model is the input of the atomic completion model. Exemplarily, the dimensions of the information of the input and the output of the atomic completion model are the same. Exemplarily, the input of the atomic completion model is assumed information of the features of atoms in the molecule, and the output of the atomic completion model is representation information of the features of atoms in the molecule. That is to say, the input of the inverse model of the atomic completion model is representation information of the features of atoms in the molecule, and the output of the inverse model of the atomic completion model is assumed information of the features of atoms in the molecule.

Because the current input information is the sample atomic feature information (that is, the representation information of the features of atoms in the sample compound molecule), step 5024 can be implemented by calling the inverse model of the atomic completion model in the molecule completion model. That is to say, the inverse model of the atomic completion model in the molecule completion model is called to perform an inverse transformation on the sample atomic feature information based on the atomic mask information to obtain the sample atomic feature hidden variable.

Exemplarily, the atomic completion model includes at least one atomic completion module, and each atomic completion module has the same structure. The aspect of this disclosure takes the atomic completion model including one atomic completion module as an example for description.

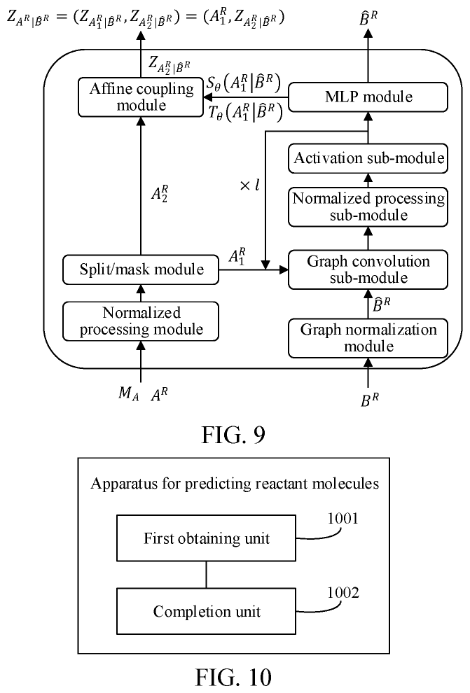

Exemplarily, the structure of the atomic completion model may be shown in FIG. 9. The atomic completion model includes a normalized processing (Actnorm) module, a split/mask module, an affine coupling module, a graph normalization (Graphnorm) module, and a transformation information obtaining module. Exemplarily, the transformation information obtaining module includes at least one reference processing module and one multilayer perceptron (MLP) module, and any reference processing module includes a graph convolution sub-module, a normalized processing (Batchnorm) sub-module, and an activation (Relu) sub-module. In FIG. 9, the number of reference processing modules is 1 (1 is an integer not less than 1). The split/mask module is configured to split the sample atomic feature information of the sample compound into two parts (the first atomic feature information and the second atomic feature information) based on the atomic mask information; the normalized processing module is configured to perform a normalized operation for each row of a matrix within each batch; the affine coupling module is configured to implement an inverse transformation of the second atomic feature information; and the transformation information obtaining module is configured to obtain the second sample transformation information.

Exemplarily, under the structure of the atomic completion model shown in FIG. 9, the process of obtaining the sample atomic feature hidden variable may be as follows: atomic mask information M_{A} and sample atomic feature information A^{R} are inputted into the atomic completion model, and are sequentially processed by the normalized processing module and the split/mask module to obtain first atomic feature information A₁^{R} and second atomic feature information A₂^{R}. The graph normalization module performs normalized processing on sample chemical bond link information B^{R} to obtain sample constraint information \hat{B}^{R}. The transformation information obtaining module including 1 reference processing modules (each reference processing module includes a graph convolution sub-module, a normalized processing sub-module, and an activation sub-module) and one MLP module is configured to process the first atomic feature information A₁^{R} and the sample constraint information B^{R} to obtain second sample transformation information S_{θ}(A₁^{R}|\hat{B}^{R}) and T_{θ}(A₁^{R}|\hat{B}^{R}), and then, the affine coupling module performs an inverse transformation on the second atomic feature information A₂^{R} through the second sample transformation information S_{θ}(A₁^{R}|\hat{B}^{R}) and T_{θ}(A₁^{R}|\hat{B}^{R}) to obtain an atomic feature hidden variable Z_{A}_{₂}_{^{R}}_{|\hat{B}}_{^{R}} of the sub-structure; and a sample atomic feature hidden variable Z_{A}_{^{R}}_{|\hat{B}}_{^{R}} is obtained based on the first atomic feature information A₁^{R} and the atomic feature hidden variable Z_{A}_{₂}_{^{R}}_{|\hat{B}}_{^{R}} of the sub-structure.

The structure of the atomic completion model shown in FIG. 9 is only an example, which is not limited in the aspect of this disclosure. That is to say, the structure of the atomic completion model may further include more or less modules.

Step 5025: Determine, by the computer device, training loss based on the sample chemical bond link hidden variable and the sample atomic feature hidden variable.

The training loss is used for measuring the prediction quality of the sample chemical bond link hidden variable and the sample atomic feature hidden variable. For example, the training loss is a value, where the larger the training loss, the worse the prediction quality of the sample chemical bond link hidden variable and the sample atomic feature hidden variable, that is, the worse the performance of the molecule completion model; and the smaller the training loss, the better the prediction quality of the sample chemical bond link hidden variable and the sample atomic feature hidden variable, that is, the better the performance of the molecule completion model.

In an exemplary aspect, the implementation process of determining the training loss based on the sample chemical bond link hidden variable and the sample atomic feature hidden variable includes the steps of: determining a first likelihood function value based on the sample chemical bond link hidden variable and the first sample transformation information; determining a second likelihood function value based on the sample atomic feature hidden variable and the second sample transformation information; determining a target likelihood function value based on the first likelihood function value and the second likelihood function value; and determining a value negatively correlated with the target likelihood function value as the training loss. For example, an opposite number of the target likelihood function value is used as the training loss, or an opposite number of a logarithmic value of the target likelihood function value is used as the training loss.

The first likelihood function value is used for measuring the probability of calling the chemical bond completion model to obtain the sample chemical bond link information on the basis of providing the sample chemical bond link hidden variable. Exemplarily, the first likelihood function value may be calculated by Formula 12:

$$log {P}_{{B}^{R}} ( {B}^{R} ) = log {P}_{{Z}_{{B}^{R}}} ( {Z}_{{B}^{R}} ) + log \vert det ( \frac{\partial {f}_{{B}^{R}}}{\partial {B}^{R}} ) \vert \qquad ( Formula 12 )$$

where the P_{B}_{^{R}}(B^{R}) represents the first likelihood function value; the B^{R} represents the sample chemical bond link information; the Z_{B}_{^{R}} represents the sample chemical bond link hidden variable; the P_{Z}_{_{B}}_{^{R}}(Z_{B}_{^{R}}) represents the probability of the sample chemical bond link hidden variable; and the det(∂f_{B}_{^{R}}/∂B^{R}) is a determinant that represents the difference between the logarithmic value of the first likelihood function value and the logarithmic value of the probability of the sample chemical bond link hidden variable, and the det((∂f_{B}_{^{R}}/∂B^{R}) is calculated based on the first sample transformation information.

The second likelihood function value is used for measuring the probability of calling the chemical bond completion model to obtain the sample atomic feature information on the basis of providing the sample atomic feature hidden variable. Exemplarily, the second likelihood function value may be calculated by Formula 13:

$$log {P}_{{A}^{R} \vert {\hat{B}}^{R}} ( {A}^{R} \vert {\hat{B}}^{R} ) = log {P}_{{z}_{{A}^{R} \vert {\hat{B}}^{R}}} ( {Z}_{{A}^{R} \vert {\hat{B}}^{R}} ) + log \vert det ( \frac{\partial {f}_{{A}^{R} \vert {B}^{R}}}{\partial {A}^{R} \vert {\hat{B}}^{R}} ) \vert \qquad ( Formula 13 )$$

where the P_{A}_{^{R}}_{|\hat{B}}_{^{R}}(A^{R}|\hat{B}^{R}) represents the second likelihood function value; the P_{Z}_{_{A}}_{^{R}}_{|\hat{B}}_{^{R}}(Z_{A}_{^{R}}_{|\hat{B}}_{^{R}}) represents the probability of the sample atomic feature hidden variable; the A^{R}|\hat{B}^{R} represents the sample atomic feature information obtained under the constraint of the \hat{B}^{R}; the Z_{A}_{^{R}}_{|\hat{B}}_{^{R}} represents the sample atomic feature hidden variable obtained under the constraint of the \hat{B}^{R}; the \hat{B}^{R} represents the sample constraint information; and the det(∂f_{A}_{^{R}}_{|B}_{^{R}}/∂_{A}_{^{R}}_{|\hat{B}}_{^{R}}) is a determinant that represents the difference between the logarithmic value of the second likelihood function value and the logarithmic value of the probability of the sample atomic feature hidden variable, and the det(∂f_{A}_{^{R}}_{|B}_{^{R}}/∂_{A}_{^{R}}_{|\hat{B}}_{^{R}}) is calculated based on the second sample transformation information.

Exemplarily, the process of obtaining the target likelihood function value based on the first likelihood function value and the second likelihood function value is shown in Formula 14:

$$log {P}_{{G}^{R}} ( {G}^{R} ) = log {P}_{{B}^{R}} ( {B}^{R} ) + log {P}_{{A}^{R} \vert {\hat{B}}^{R}} ( {A}^{R} \vert {\hat{B}}^{R} ) \qquad ( Formula 14 )$$

where the G^{R} represents the sample compound molecule; the B^{R} represents the sample chemical bond link information; the A^{R}|\hat{B}^{R} represents the sample atomic feature information; the P_{B}_{^{R}}(B^{R}) represents the first likelihood function value; the P_{A}_{^{R}}_{|\hat{B}}_{^{R}}(A^{R}|\hat{B}^{R}) represents the second likelihood function value; and the P_{G}_{^{R}}(G^{R}) represents the target likelihood function value.

Exemplarily, the process of obtaining the target likelihood function value may be as follows:
input: Information G^{R}=(A^{R}, B^{R}) of the sample compound molecule and mask information M thereof (including atomic mask information and chemical bond mask information), an inverse model f_{A}_{^{R}}_{|B}_{^{R}} of the atomic completion model, and an inverse model f_{B}_{^{R}} of the chemical bond completion model, where the A^{R} represents the sample atomic feature information, and the B^{R} represents the sample chemical bond link information;
1. \hat{B}′^{R}=GLOW(B^{R})//use a GLOW module (an information processing module) to process the sample chemical bond link information B^{R} to obtain chemical bond link information B′R after processing
2. Z_{B}_{^{R}}=f_{B}_{^{R}}(\hat{B}′^{R})//call the inverse model f_{B}_{^{R}} of the chemical bond completion model to obtain a chemical bond link hidden variable Z_{B}_{^{R}} based on the chemical bond link information \hat{B}′^{R} after processing, where this process considers the chemical bond mask information in the mask information M

$$3. log {P}_{{B}^{R}} ( {B}^{R} ) = log {P}_{{Z}_{{B}^{R}}} ( {Z}_{{B}^{R}} ) + log \vert det ( \frac{\partial {f}_{{B}^{R}}}{\partial {B}^{R}} ) \vert$$

//obtain a first likelihood function value log P_{B}_{^{R}}(B^{R}) based on the chemical bond link information Z_{B}_{^{R}} and a determinant det(∂f_{B}_{^{R}}/∂_{B}_{^{R}})
4. \hat{B}^{R}=Graphnorm(B^{R})//use a Graphnorm module (a graph normalization module) to perform normalized processing on the sample chemical bond link information B^{R} to obtain sample constraint information \hat{B}^{R}
5. Z_{A}_{^{R}}_{|\hat{B}}_{^{R}}=f_{A}_{^{R}}_{|B}_{^{R}}(A^{R}|\hat{B}^{R})//call the inverse model f_{A}_{^{R}}_{|B}_{^{R}} of the atomic completion model to obtain an atomic feature hidden variable Z_{A}_{^{R}}_{|\hat{B}}_{^{R}} based on the sample atomic feature information A^{R} and the sample constraint information \hat{B}^{R}, where this process considers the atomic mask information in the mask information M

$$6. log {P}_{{A}^{R} \vert {\hat{B}}^{R}} ( {A}^{R} \vert {\hat{B}}^{R} ) = log {P}_{{Z}_{{A}^{R} \vert {\hat{B}}^{R}}} ( {Z}_{{A}^{R} \vert {\hat{B}}^{R}} ) + log \vert det ( \frac{\partial {f}_{{A}^{R} \vert {B}^{R}}}{\partial {A}^{R} \vert {\hat{B}}^{R}} ) \vert$$

//obtain a second likelihood function value log P_{A}_{^{R}}_{|\hat{B}}_{^{R}}(A^{R}|\hat{B}^{R}) based on the atomic feature hidden variable Z_{A}_{^{R}}_{|\hat{B}}_{^{R}} and a determinant det(∂f_{A}_{^{R}}_{|B}_{^{R}}/∂_{A}_{^{R}}_{|\hat{B}}_{^{R}})
7. Z_{G}_{^{R}}=(Z_{A}_{^{R}}_{|\hat{B}}_{^{R}}, Z_{B}_{^{R}})//take the information including the atomic feature hidden variable Z_{A}_{^{R}}_{|\hat{B}}_{^{R}} and the chemical bond link hidden variable Z_{B}_{^{R}} as a sample hidden variable Z_{G}_{^{R}}
8. log P_{G}_{^{R}}(G^{R})=log P_{B}_{^{R}}(B^{R})+log P_{A}_{^{R}}_{|\hat{B}}_{^{R}}(A^{R}|\hat{B}^{R})//obtain a logarithmic value of a target likelihood function value P_{G}_{^{R}}(G^{R}) based on the first likelihood function value P_{B}_{^{R}}(B^{R}) and the second likelihood function value P_{A}_{^{R}}_{|\hat{B}}_{^{R}}(A^{R}|\hat{B}^{R})
output: Z_{G}_{^{R}}, log P_{G}_{^{R}}(G^{R}).

The process of determining the training loss based on step 5021 to step 5025 is only an exemplary implementation process, which is not limited in the aspect of this disclosure. In an exemplary aspect, the implementation manner of obtaining the training loss based on the sample compound molecule, the sample molecule to be completed and the molecule completion model may also be as follows: the computer device calls the molecule completion model to complete the sample molecule to be completed to obtain a completion result, and determines a predicted completion molecule based on the completion result; and determines the training loss based on the difference between the predicted completion molecule and the sample compound molecule.

The implementation process of calling the molecule completion model to complete the sample molecule to be completed refers to step 203 in the aspect shown in FIG. 2, and will not be repeated here. The predicted completion molecule can be obtained based on the completion result obtained by calling the molecule completion model to complete the sample molecule to be completed. The predicted completion molecule is a molecule after completion of the sample molecule to be completed predicted by the molecule completion model. The sample compound molecule is a real molecule after completion of the sample molecule to be completed. The training loss for providing supervision information for the updating of the model parameters of the molecule completion model can be obtained based on the difference between the predicted completion molecule and the sample compound molecule.

The aspect of this disclosure does not limit the manner of measuring the difference between two molecules. Exemplarily, the difference between two molecules may be determined based on the difference of atoms in two molecules (difference in atomic number, difference in atomic type, difference in atomic feature, and the like) and the difference between chemical bonds (difference in chemical bond number, difference in chemical bond type, difference in chemical bond feature, and the like). Exemplarily, molecule features of two molecules are extracted, and the difference between the two molecule features is used as the difference between the two molecules. Exemplarily, molecule features of two molecules may be extracted by calling a molecule feature extraction model. Exemplarily, two molecule features are vectors or matrices with the same dimension, and the difference between the two molecule features may be determined based on the difference between elements at corresponding positions in the two molecule features. Exemplarily, a similarity between two molecule features may also be calculated, and a value negatively correlated with the similarity (such as an opposite number of the similarity) is used as the difference between the two molecule features.

In either case, after the training loss is determined, the model parameters of the molecule completion model are updated based on the training loss. Exemplarily, the process of updating the model parameters of the molecule completion model based on the training loss may be implemented based on a gradient descent method, that is, an update gradient of the model parameters of the molecule completion model is obtained based on the training loss, and the model parameters of the molecule completion model are updated based on the update gradient. Exemplarily, in a case of determining the training loss based on step 5021 to step 5025, the process of updating the model parameters of the molecule completion model based on the training loss may also refer to a process of updating the inverse model of the molecule completion model based on the training loss.

Exemplarily, the model training process is an iterative process. After the model parameters of the molecule completion model are updated based on the training loss, a molecule completion model which is trained once is obtained; whether the current training process mects a target termination condition is judged; if the current training process meets the target termination condition, the molecule completion model which is trained once may be used as the molecule completion model after training; and if the current training process does not meet the target termination condition, new training loss may be obtained by referring to step 501 and step 502, then the model parameters of the currently obtained molecule completion model are updated through the new training loss, and so on, until the current training process meets the target termination condition, and the molecule completion model obtained when the current training process meets the target termination condition is used as the molecule completion model after training. The sample compound molecule and sample molecule to be completed for obtaining the new training loss may be partially or completely changed, or may be not changed.

Mecting the target termination condition may be set according to experiences or flexibly adjusted according to application scenarios, which is not limited in the aspect of this disclosure. In an exemplary aspect, meeting the target termination condition may mean that the training loss converges, the training loss is less than a second loss threshold, or the number of updates of the model parameters reaches a second number threshold. The second loss threshold and the second number threshold may be set according to experiences or flexibly adjusted according to application scenarios, which are not limited in the aspect of this disclosure.

In an exemplary aspect, the process of obtaining the molecule completion model by training may be a curriculum learning process. In this case, the molecule completion model can be gradually trained by constructing tasks from easy to difficult. Exemplarily, the difficulty of a task may be measured based on the complexity of the masked sub-structure in the sample compound molecule or the masking ratio of the masked sub-structure in the sample compound molecule. If the complexity of the masked sub-structure in the sample compound molecule is relatively low or the masking ratio of the masked sub-structure in the sample compound molecule is relatively low, the model may receive more information, and the molecule completion task is relatively easy; and if the complexity of the masked sub-structure in the sample compound molecule is relatively high or the masking ratio of the masked sub-structure in the sample compound molecule is relatively high, the model may receive less information, and the molecule completion task is relatively difficult. The training effect of the molecule completion model can be gradually improved by gradually training the molecule completion model through tasks from casy to difficult. Certainly, in some aspects, the molecule completion model may also be trained directly by complex tasks (such as tasks constructed under sub-graph-level mask solutions), which is not limited in the aspect of this disclosure.

Exemplarily, in a case that the molecule completion model is gradually trained through tasks from easy to difficult, meeting the target termination condition may also refer to completing the training of the molecule completion model based on a task of the maximum difficulty. Exemplarily, training the molecule completion model based on a task of any difficulty refers to training the molecule completion model based on sample data of the any difficulty. For example, the sample data is a sample compound molecule and a sample molecule to be completed thereof matched with the any difficulty. Completing the training of the molecule completion model based on the task of any difficulty may mean that in the process of training the molecule completion model based on the sample data of any difficulty, the loss reaches convergence, or the loss is less than a certain loss threshold, or the training number reaches a certain number threshold.

In an exemplary aspect, the process of obtaining the molecule completion model by training may also be a process of first performing pre-training and then performing fine adjustment, where the sample data for pre-training is obtained based on a compound molecule in any unlabeled compound molecule data set, and the sample data for fine adjustment is obtained based on a product molecule in a known synthesis reaction. In this case, meeting the target termination condition may refer to completing the fine adjustment of the molecule completion model. For example, in the process of training the molecule completion model based on the sample data for fine adjustment, the loss reaches convergence, or the loss is less than a certain loss threshold, or the training number reaches a certain number threshold.

Some common parameters used in the model training process, such as a learning rate, an iteration cycle (epoch) and a batch size, may be set according to experiences or flexibly adjusted according to the computing capability of the computer device or application scenarios, which are not limited in the aspect of this disclosure.

The training process of the molecule completion model provided in the aspect of this disclosure can first perform pre-training on an unlabeled large data set. This operation indirectly achieves the data augmentation to expand more available effective information, which can improve the generalization ability of the molecule completion model to adapt to wider reactant molecule prediction scenarios, thus improving the prediction reliability and prediction accuracy of the reactant molecule. Because a pre-trained molecule completion task is very related to a completion task of a molecule to be completed in a reactant molecule prediction process, after pre-training, the molecule completion model can be finely adjusted on the basis of a data set including known synthesis reactions (such as an inverse synthesis data set) to enable the model to have a stronger generalization ability. Exemplarily, completion tasks of the molecule to be completed in the reactant molecule prediction process may be considered as some special cases of general molecule completion tasks.

Exemplarily, the aspect of this disclosure uses the molecule reconstruction represented based on graphs as a self-supervised learning task. This solution can expand previous molecular self-supervised learning tasks and better apply the idea of “Mask and Fill” to the field of graph structure data. A self-supervised learning strategy is applied to the field of prediction of reactant molecules. A model learns self-supervised tasks (such as molecular structure completion) on a molecular large data set, then fine adjustment is performed on an inverse synthesis data set, and finally, a reactant molecule can be directly predicted through a provided product molecule, thus deriving a synthesis path. The model trained by this manner can improve the prediction ability of reactant molecules and break through data bottlenecks, and has a stronger generalization ability. In addition, the aspect of this disclosure can use a flow-based generative model to implement the completion of molecules. The flow-based generative model is a non-autoregressive generative model which can be generated once. Compared with an autoregressive model, the flow-based generative model has higher generation efficiency and faster inference speed and can achieve similar or higher prediction accuracy. Moreover, the flow-based generative model can provide a likelihood function value of a prediction result and has better interpretability.

According to the method for training molecule completion models provided in the aspect of this disclosure, the molecule completion model is trained based on the sample compound molecule and the sample molecule to be completed. The sample molecule to be completed is obtained by masking the sub-structure in the sample compound molecule, that is, the data for the training process of the molecule completion model is the data obtained on the basis of the sample compound molecule, this training process is a self-supervised training process based on the sample compound molecule, and this self-supervised training process does not require attention to whether the sample compound molecule is a compound in a known synthesis reaction, so that this self-supervised training process is not limited by the known synthesis reaction, and the molecule completion model trained by the training process has a stronger generalization ability, which is beneficial for expanding adaptation scenarios so as to improve the prediction reliability and prediction accuracy of the reactant molecule.

Referring to FIG. 10, an aspect of this disclosure provides an apparatus for predicting reactant molecules, arranged in a computer device. The apparatus includes:
a first obtaining unit 1001, configured to obtain a product molecule, and break bonds of the product molecule to obtain a molecule to be completed, the product molecule referring to any compound molecule of a reactant molecule to be predicted; and
a completion unit 1002, configured to call a molecule completion model to complete the molecule to be completed to obtain a completion result, and determine a reactant molecule of the product molecule based on the completion result,
where the molecule completion model is obtained by training based on a sample compound molecule and a sample molecule to be completed, and the sample molecule to be completed is obtained by masking a sub-structure in the sample compound molecule.

In a possible implementation, the completion unit 1002 is configured to determine a target atomic feature hidden variable based on atomic feature information of the molecule to be completed, the target atomic feature hidden variable being an atomic feature hidden variable of the molecule after completion; determine a target chemical bond link hidden variable based on chemical bond link information of the molecule to be completed, the target chemical bond link hidden variable being a chemical bond link hidden variable of the molecule after completion; call the molecule completion model to transform the target chemical bond link hidden variable to obtain target chemical bond link information, and transform the target atomic feature hidden variable to obtain target atomic feature information; and determine a completion result of the molecule to be completed based on the target chemical bond link information and the target atomic feature information.

In a possible implementation, the first obtaining unit 1001 is configured to obtain graph structure information of the product molecule; predict breakage probabilities of chemical bonds in the product molecule based on the graph structure information, and determine the chemical bonds with the breakage probabilities meeting reference conditions as breakage chemical bonds in the product molecule; and break bonds of the product molecule based on the breakage chemical bonds to obtain the molecule to be completed.

According to the apparatus for predicting reactant molecules provided in the aspect of this disclosure, the prediction process of the reactant molecule is implemented by the molecule completion model, and the molecule completion model is obtained by training based on the sample compound molecule and the sample molecule to be completed. The sample molecule to be completed is obtained by masking the sub-structure in the sample compound molecule, that is, the data for the training process of the molecule completion model is the data obtained on the basis of the sample compound molecule, this training process is a self-supervised training process based on the sample compound molecule, and this self-supervised training process does not require attention to whether the sample compound molecule is a compound in a known synthesis reaction, so that this self-supervised training process is not limited by the known synthesis reaction, and the molecule completion model trained by the training process has a stronger generalization ability, which is beneficial for expanding adaptation scenarios so as to improve the prediction reliability and prediction accuracy of the reactant molecule.

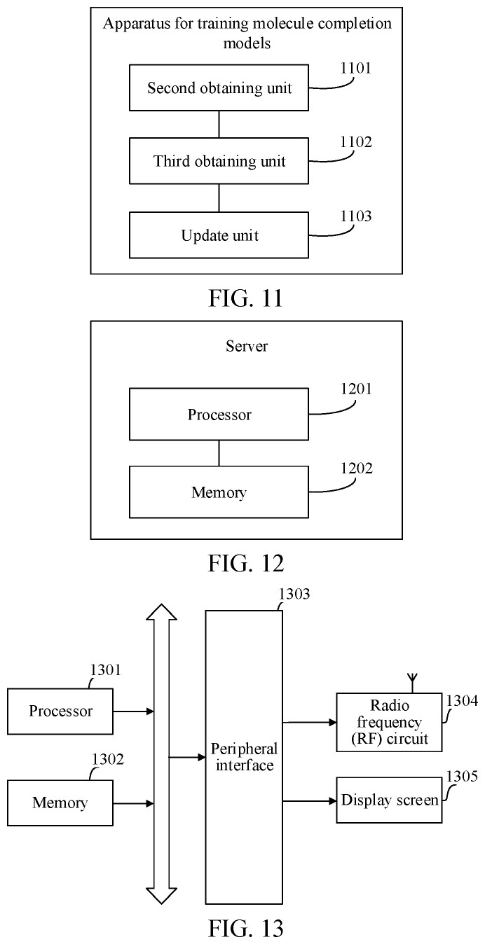

Referring to FIG. 11, an aspect of this disclosure provides an apparatus for training molecule completion models. The apparatus includes:
a second obtaining unit 1101, configured to obtain a sample compound molecule and a sample molecule to be completed, the sample molecule to be completed being obtained by masking a sub-structure in the sample compound molecule;
a third obtaining unit 1102, configured to determine training loss based on the sample compound molecule, the sample molecule to be completed, and a molecule completion model; and
an update unit 1103, configured to update model parameters of the molecule completion model based on the training loss to obtain a molecule completion model after training.

In a possible implementation, the third obtaining unit 1102 is configured to obtain sample atomic feature information and sample chemical bond link information of the sample compound molecule; determine atomic mask information and chemical bond mask information based on a difference between the sample compound molecule and the sample molecule to be completed; call the molecule completion model to perform an inverse transformation on the sample chemical bond link information based on the chemical bond mask information to obtain a sample chemical bond link hidden variable, and perform an inverse transformation on the sample atomic feature information based on the atomic mask information to obtain a sample atomic feature hidden variable; and determine the training loss based on the sample chemical bond link hidden variable and the sample atomic feature hidden variable.

In a possible implementation, the third obtaining unit 1102 is configured to call the molecule completion model to determine first chemical bond link information and second chemical bond link information based on the chemical bond mask information and the sample chemical bond link information, the first chemical bond link information being chemical bond link information of the sample molecule to be completed, and the second chemical bond link information being chemical bond link information of the sub-structure; perform an inverse transformation on the second chemical bond link information based on the first chemical bond link information to obtain a chemical bond link hidden variable of the sub-structure; and determine the sample chemical bond link hidden variable based on the first chemical bond link information and the chemical bond link hidden variable of the sub-structure.

In a possible implementation, the third obtaining unit 1102 is configured to determine first atomic feature information and second atomic feature information based on the atomic mask information and the sample atomic feature information, the first atomic feature information being atomic feature information of the sample molecule to be completed, and the second atomic feature information being atomic feature information of the sub-structure; perform an inverse transformation on the second atomic feature information based on the first atomic feature information to obtain an atomic feature hidden variable of the sub-structure; and determine the sample atomic feature hidden variable based on the first atomic feature information and the atomic feature hidden variable of the sub-structure.

In a possible implementation, the third obtaining unit 1102 is configured to call the molecule completion model to complete the sample molecule to be completed to obtain a completion result, and determine a predicted completion molecule based on the completion result; and determine the training loss based on a difference between the predicted completion molecule and the sample compound molecule.

In a possible implementation, the sub-structure is a structure belonging to a candidate structure set in the sample compound molecule, and the candidate structure set is a set of structures with confidences meeting selection conditions.

According to the apparatus for training molecule completion models provided in the aspect of this disclosure, the molecule completion model is trained based on the sample compound molecule and the sample molecule to be completed. The sample molecule to be completed is obtained by masking the sub-structure in the sample compound molecule, that is, the data for the training process of the molecule completion model is the data obtained on the basis of the sample compound molecule, this training process is a self-supervised training process based on the sample compound molecule, and this self-supervised training process does not require attention to whether the sample compound molecule is a compound in a known synthesis reaction, so that this self-supervised training process is not limited by the known synthesis reaction, and the target molecule completion model trained by the training process has a stronger generalization ability, which is beneficial for expanding adaptation scenarios so as to improve the prediction reliability and prediction accuracy of the reactant molecule.

When the apparatus provided in the foregoing aspect implements the functions of the apparatus, only division of the foregoing functional units is taken as an example for description. In practical applications, the foregoing functions may be allocated to and completed by different functional units according to requirements. That is, an internal structure of the device is divided into different functional units to complete all or some of the functions described above. In addition, the apparatus provided by the above aspects belong to a same conception as the method aspect. For details of the specific implementation process, reference can be made to the method aspect, and details will not be repeated here.

In an exemplary aspect, a computer device is further provided. The computer device includes a processor (e.g., processing circuitry) and a memory (e.g., a non-transitory computer-readable storage medium). The memory stores at least one computer program. The at least one computer program is loaded and executed by one or more processors, so that the computer device implements any one of the foregoing methods for predicting reactant molecules or training methods for molecule completion models. The computer device may be a server or a terminal. Then, the structures of the server and the terminal are introduced respectively.

FIG. 12 is a schematic structural diagram of a server provided in an aspect of this disclosure. The server may vary greatly due to different configurations or performances, and may include one or a plurality of processors (such as central processing units (CPUs)) 1201 and one or a plurality of memories 1202, where the one or plurality of memories 1202 store at least one computer program, and the at least one computer program is loaded and executed by the one or plurality of processors 1201, so that the server implements the methods for predicting reactant molecules or training methods for molecule completion models provided in the foregoing method aspects. Certainly, the server may further include components such as a wired or wireless network interface, a keyboard, and an input/output (I/O) interface to facilitate input and output. The server may further include other components configured to implement device functions. Details are not further described herein.

FIG. 13 is a schematic structural diagram of a terminal provided in an aspect of this disclosure. The terminal may be: a PC, a mobile phone, a smart phone, a PDA, a wearable device, a PPC, a tablet computer, a smart car machine, a smart television, a smart speaker, a smart voice interaction device, a smart home appliance, an on board terminal, a VR device, or an AR device. The terminal may also be referred to as another name such as user equipment, a portable terminal, a laptop terminal, or a desktop terminal. Generally, the terminal includes: a processor 1301 and a memory 1302.

The processor 1301 may include one or a plurality of processing cores, for example, a 4-core processor or an 8-core processor. The processor 1301 may be implemented in at least one hardware form of digital signal processing (DSP), a field-programmable gate array (FPGA), and a programmable logic array (PLA).

The memory 1302 may include one or a plurality of computer-readable storage media. The computer-readable storage medium may be non-transient or non-transitory. The memory 1302 may further include a high-speed random access memory and a non-volatile memory, for example, one or a plurality of disk storage devices or flash storage devices. In some aspects, the non-transient computer-readable storage medium in the memory 1302 is configured to store at least one instruction, and the at least one instruction is executed by the processor 1301, so that the terminal implements the methods for predicting reactant molecules or training methods for molecule completion models provided in the method aspects of this disclosure.

In some aspects, the terminal may further include: a peripheral interface 1303 and at least one peripheral. The processor 1301, the memory 1302, and the peripheral interface 1303 may be connected through a bus or a signal cable. Each peripheral may be connected to the peripheral interface 1303 through a bus, a signal cable, or a circuit board. Specifically, the peripheral includes: at least one of a radio frequency (RF) circuit 1304 and a display screen 1305.

The peripheral interface 1303 may be configured to connect at least one peripheral related to I/O to the processor 1301 and the memory 1302. In some aspects, the processor 1301, the memory 1302, and the peripheral interface 1303 are integrated on a same chip or circuit board. In some other aspects, any one or two of the processor 1301, the memory 1302 and the peripheral interface 1303 may be implemented on a single chip or circuit board. This is not limited in this aspect.

The RF circuit 1304 is configured to receive and transmit an RF signal, also referred to as an electromagnetic signal. The RF circuit 1304 communicates with a communication network and other communication devices through the electromagnetic signal. The RF circuit 1304 transforms an electric signal into an electromagnetic signal for transmission, or transforms a received electromagnetic signal into an electric signal.

The display screen 1305 is configured to display a user interface (UI). The UI may include a graph, a text, an icon, a video, and any combination thereof. In a case that the display screen 1305 is a touch display screen, the display screen 1305 further has a capability of acquiring a touch signal on or above a surface of the display screen 1305. The touch signal may be inputted to the processor 1301 as a control signal for processing. In this case, the display screen 1305 may be further configured to provide a virtual button and/or a virtual keyboard that are/is also referred to as a soft button and/or a soft keyboard.

A person skilled in the art may understand that the structure shown in FIG. 13 constitutes no limitation on the terminal, and the terminal may include more or fewer components than those shown in the figure, or some components may be combined, or different component layouts may be used.

In an exemplary aspect, a computer-readable storage medium is further provided. The computer-readable storage medium stores at least one computer program. The at least one computer program is loaded and executed by a processor of a computer device, so that the computer implements any one of the foregoing methods for predicting reactant molecules or training methods for molecule completion models.

In a possible implementation, the foregoing computer-readable storage medium may be a read-only memory (ROM), a random access memory (RAM), a compact disc read-only memory (CD-ROM), a magnetic tape, a floppy disk, an optical data storage device, or the like.

In an exemplary aspect, a computer program product is further provided. The computer program product includes a computer program or a computer instruction. The computer program or computer instruction is loaded and executed by a processor, so that the computer implements any one of the foregoing methods for predicting reactant molecules or training methods for molecule completion models.

The information (including but not limited to user equipment information, user personal information, and the like), data (including but not limited to data used for analysis, stored data, displayed data, and the like), and signals involved in this disclosure are all authorized by users or fully authorized by all parties, and the collection, use and processing of the relevant data need to comply with relevant laws, regulations and standards of relevant countries and regions. For example, the product molecules and the like involved in this disclosure are obtained in a case of full authorization.

It is to be understood that the term “a plurality of” mentioned herein means two or more. The term “and/or” describes an association relationship between associated objects and represents that three relationships may exist. For example, A and/or B may represent the following three cases: only A exists, both A and B exist, and only B exists. The character “/” generally represents an “or” relationship between the associated objects before and after it.

The term module (and other similar terms such as unit, submodule, etc.) in this disclosure may refer to a software module, a hardware module, or a combination thereof. A software module (e.g., computer program) may be developed using a computer programming language. A hardware module may be implemented using processing circuitry and/or memory. Each module can be implemented using one or more processors (or processors and memory). Likewise, a processor (or processors and memory) can be used to implement one or more modules. Moreover, each module can be part of an overall module that includes the functionalities of the module.

The foregoing disclosure includes some exemplary embodiments of this disclosure which are not intended to limit the scope of this disclosure. Other embodiments shall also fall within the scope of this disclosure.

What is claimed is:

1. A method for predicting reactant molecules, the method comprising:
obtaining a product molecule, and selecting bonds of the product molecule to be broken to obtain a molecule to be completed, the product molecule defining a compound molecule of a reactant molecule to be predicted; and
applying a molecule completion model to complete the molecule to be completed to obtain a completion result indicating the reactant molecule of the product molecule based on the molecule to be completed,
wherein the molecule completion model is obtained by training based on sample compound molecules and sample molecules to be completed obtained by masking sub-structures in the sample compound molecules.

2. The method according to claim 1, wherein the applying the molecule completion model comprises:
determining an atomic feature hidden variable for completing the molecule to be completed based on atomic feature information of the molecule to be completed;
determining a chemical bond link hidden variable for completing the molecule to be completed based on chemical bond link information of the molecule to be completed;
applying the molecule completion model to transform the chemical bond link hidden variable to obtain chemical bond link information, and transform the atomic feature hidden variable to obtain atomic feature information; and
determining the completion result of the molecule to be completed based on the chemical bond link information and the atomic feature information.

3. The method according to claim 2, wherein the transforming the chemical bond link hidden variable to obtain chemical bond link information comprises:
obtaining reference chemical bond link information of the molecule to be completed and a reference chemical bond link hidden variable of a missing structure based on the chemical bond link hidden variable;
transforming the reference chemical bond link hidden variable of the missing structure based on the reference chemical bond link information of the molecule to be completed to obtain reference chemical bond link information of the missing structure; and
determining the chemical bond link information based on the reference chemical bond link information of the molecule to be completed and the reference chemical bond link information of the missing structure.

4. The method according to claim 2, wherein the transforming the atomic feature hidden variable to obtain target atomic feature information comprises:
obtaining reference atomic feature information of the molecule to be completed and a reference atomic feature hidden variable of a missing structure based on the atomic feature hidden variable;
transforming the reference atomic feature hidden variable of the missing structure based on the reference atomic feature information of the molecule to be completed to obtain reference atomic feature information of the missing structure; and
determining the atomic feature information based on the reference atomic feature information of the molecule to be completed and the reference atomic feature information of the missing structure.

5. The method according to claim 1, wherein the selecting the bonds of the product molecule to be broken comprises:
obtaining graph structure information of the product molecule;
predicting breakage probabilities of chemical bonds in the product molecule based on the graph structure information;
determining the chemical bonds with the breakage probabilities meeting reference conditions as breakage chemical bonds in the product molecule; and
selecting the breakage chemical bonds as the bonds of the product molecule to be broken to obtain the molecule to be completed.

6. The method according to claim 5, wherein
the graph structure information of the product molecule comprises atomic feature information and chemical bond link information of the product molecule, the atomic feature information comprises sub-feature information of each atom in the product molecule, and the chemical bond link information comprises sub-feature information of each chemical bond in the product molecule; and
the obtaining the graph structure information of the product molecule comprises:
obtaining attribute information of each atom in the product molecule, and performing feature extraction on the attribute information of each atom to obtain sub-feature information of each atom; and
obtaining attribute information of each chemical bond in the product molecule, and performing feature extraction on the attribute information of each chemical bond to obtain sub-feature information of each chemical bond.

7. The method according to claim 5, wherein the predicting the breakage probabilities of the chemical bonds in the product molecule comprises:
applying a graph neural network model to extract features for predicting breakage probabilities of the chemical bonds in the product molecule based on the graph structure information, and predicting the breakage probabilities of the chemical bonds in the product molecule based on the features.

8. The method according to claim 7, wherein a training process of the graph neural network model comprises:
obtaining graph structure information of a training compound molecule and a standard breakage probability of chemical bonds in the training compound molecule;
applying a training graph neural network model to predict a training breakage probability of the chemical bonds in the training compound molecule based on the graph structure information of the training compound molecule;
determining reference loss based on a difference between the standard breakage probability and the training breakage probability;
updating model parameters of the training graph neural network model based on the reference loss; and
determining the training graph neural network model as the graph neural network model in response to the training process meeting a first termination condition.

9. The method according to claim 1, further comprising determining the reactant molecule of the product molecule based on the completion result by one of:
determining graph structure information of the reactant molecule when the completion result is graph structure information of the molecule after completion; or
determining a missing structure for completing the molecule to be completed according to indication information of the missing structure when the completion result is the indication information, linking the missing structure with the molecule to be completed, and determining a result of the linking as the reactant molecule.

10. The method according to claim 9, wherein
the indication information comprises a classification result of the missing structure in the molecule to be completed and a link position prediction result, the classification result comprises a matching probability of the missing structure in the molecule to be completed and a single atom level structure and a single chemical bond level structure, and the link position prediction result indicates a position in the molecule to be completed to be linked with the missing structure; and
the determining the missing structure comprises:
determining a type of the missing structure in the molecule to be completed according to the classification result, and determining a position in the molecule to be completed to be linked with the missing structure according to the link position prediction result; and
determining the reactant molecule by linking the structure of the type with the molecule to be completed at the position in the molecule to be completed to be linked with the missing structure.

11. A method for training molecule completion models, the method comprising:
obtaining a sample compound molecule and a sample molecule to be completed, the sample molecule to be completed being obtained by masking a sub-structure in the sample compound molecule;
determining a training loss based on the sample compound molecule, the sample molecule to be completed, and a molecule completion model; and
updating model parameters of the molecule completion model based on the training loss to obtain a trained molecule completion model.

12. The method according to claim 11, wherein the determining the training loss comprises:
obtaining sample atomic feature information and sample chemical bond link information of the sample compound molecule;
determining atomic mask information and chemical bond mask information based on a difference between the sample compound molecule and the sample molecule to be completed;
applying the molecule completion model to perform an inverse transformation on the sample chemical bond link information based on the chemical bond mask information to obtain a sample chemical bond link hidden variable;
applying the molecule completion model to perform an inverse transformation on the sample atomic feature information based on the atomic mask information to obtain a sample atomic feature hidden variable; and
determining the training loss based on the sample chemical bond link hidden variable and the sample atomic feature hidden variable.

13. The method according to claim 12, wherein the applying the molecule completion model to perform the inverse transformation on the sample chemical bond link information comprises:
applying the molecule completion model to determine first chemical bond link information of the sample molecule to be completed and second chemical bond link information of the sub-structure based on the chemical bond mask information and the sample chemical bond link information;
performing an inverse transformation on the second chemical bond link information based on the first chemical bond link information to obtain a chemical bond link hidden variable of the sub-structure; and
determining the sample chemical bond link hidden variable based on the first chemical bond link information and the chemical bond link hidden variable.

14. The method according to claim 12, wherein the applying the molecule completion model to perform the inverse transformation on the sample atomic feature information comprises:
applying the molecule completion model to determine first atomic feature information of the sample molecule to be completed and second atomic feature information of the sub-structure based on the atomic mask information and the sample atomic feature information;
performing an inverse transformation on the second atomic feature information based on the first atomic feature information to obtain an atomic feature hidden variable of the sub-structure; and
determining the sample atomic feature hidden variable based on the first atomic feature information and the atomic feature hidden variable.

15. The method according to claim 12, wherein the determining the training loss based on the sample chemical bond link hidden variable and the sample atomic feature hidden variable comprises:
determining a first likelihood function value based on the sample chemical bond link hidden variable and first sample transformation information;
determining a second likelihood function value based on the sample atomic feature hidden variable and second sample transformation information;
determining a target likelihood function value based on the first likelihood function value and the second likelihood function value; and
determining a value negatively correlated with the target likelihood function value as the training loss.

16. The method according to claim 11, wherein the determining the training loss comprises:
applying the molecule completion model to complete the sample molecule to be completed to obtain a completion result, and determining a predicted completion molecule based on the completion result; and
determining the training loss based on a difference between the predicted completion molecule and the sample compound molecule.

17. The method according to claim 11, wherein the sub-structure is a structure belonging to a candidate structure set in the sample compound molecule, and the candidate structure set is a set of structures meeting selection conditions.

18. An apparatus for predicting reactant molecules, the apparatus comprising:
processing circuitry configured to
obtain a product molecule, and select bonds of the product molecule to be broken to obtain a molecule to be completed, the product molecule defining a compound molecule of a reactant molecule to be predicted; and
apply a molecule completion model to complete the molecule to be completed to obtain a completion result indicating the reactant molecule of the product molecule based on the molecule to be completed,
wherein the molecule completion model is obtained by training based on sample compound molecules and sample molecules to be completed obtained by masking sub-structures in the sample compound molecules.

19. The apparatus according to claim 18, wherein the processing circuitry is further configured to:
determine an atomic feature hidden variable for completing the molecule to be completed based on atomic feature information of the molecule to be completed;
determine a chemical bond link hidden variable for completing the molecule to be completed based on chemical bond link information of the molecule to be completed;
apply the molecule completion model to transform the chemical bond link hidden variable to obtain chemical bond link information, and transform the atomic feature hidden variable to obtain atomic feature information; and
determine the completion result of the molecule to be completed based on the chemical bond link information and the atomic feature information.

20. The apparatus according to claim 19, wherein the processing circuitry is further configured to:
obtain reference chemical bond link information of the molecule to be completed and a reference chemical bond link hidden variable of a missing structure based on the chemical bond link hidden variable;
transform the reference chemical bond link hidden variable of the missing structure based on the reference chemical bond link information of the molecule to be completed to obtain reference chemical bond link information of the missing structure; and
determine the chemical bond link information based on the reference chemical bond link information of the molecule to be completed and the reference chemical bond link information of the missing structure.
